(19) United States
(12) Patent Application Publication
(10) Pub. No.: US 2023/0203495 A1
(43) Pub. Date: Jun. 29, 2023

(54) FIDGETIN-LIKE 2 AS A TARGET TO ENHANCE SKIN GRAFT HEALING

(71) Applicant: MICROCURES, INC., Santa Cruz, CA (US)

(72) Inventors: David James SHARP, Scarsdale, NY (US); Brian O'ROURKE, Rockville Centre, NY (US)

(73) Assignee: MICROCURES, INC., Santa Cruz, CA (US)

(21) Appl. No.: 17/802,905

(22) PCT Filed: Feb. 26, 2021
(86) PCT No.: PCT/US2021/020108

§ 371 Date: Aug. 26, 2022

Related U.S. Application Data
(60) Provisional application No. 62/982,484, filed on Feb. 27, 2020.

(51) Int. Cl. C12N 15/113; A61P 17/02

(52) U.S. Cl. CPC C12N 15/1137; A61P 17/02; C12N 2310/14; C12N 2310/531; C12N 2320/30

(57) ABSTRACT

Methods of accelerating or improving the healing of a skin graft or skin grafting site in a subject are provided comprising administering to the subject an amount of an inhibitor of fidgetin-like 2.

### CROSS-REFERENCE TO RELATED APPLICATION

This application claims priority to U.S. Provisional Patent Application 62/982,484, filed Feb. 27, 2020, which is incorporated by reference herein in its entirety.

### INCORPORATION OF SEQUENCE LISTING

The “.txt” Sequence Listing filed with this application by EFS and which is entitled P-592879-US-SQL-updated-06MAR23_ST25.txt, is 46.2 kilobytes in size and which was created on Mar. 9, 2023, is hereby incorporated by reference. The sequence listing submitted herewith is identical to the sequence listing forming part of the international application.

### BACKGROUND

Burns suffered by US military service personnel have typically accounted for 5 to 20% of combat casualties in recent conflicts. Operations in Iraq and Afghanistan have produced burn wounds with increasing injury severity scores and have become a more common injury due to the prevalence of the improvised explosive device (IED). In fact, IEDs caused 69.6% of burn injuries reported between April 2003 and May 2005. The enemy's success using IEDs makes it likely that these weapons will be employed against US forces in future conflicts. In addition, compared to civilian patients, combat casualties with burns are characterized by a higher percentage of full-thickness burns and larger total body surface area (TBSA). The resultant injuries will make burn treatment a significant challenge for military medical professionals moving forward. Novel approaches to burn wound management will speed recovery, decrease patient discomfort, increase return to duty rates, lower healthcare costs, and preserve the fighting force. Reducing the potential of scar formation will have a great impact on the functional outcome (range of motion) and psychological well-being (cosmesis) of these patients.

Within the civilian population, the American Burn Association estimates that nearly 500,000 individuals per year receive medical treatment for burn injuries; 40,000 of whom will require hospitalization. The associated costs for acute burn treatment and hospitalization are estimated to be $10 billion per year. Long-term treatments for scarring account for approximately $12 billion dollars per year. With fewer than 200 burn centers in the country, there is a tremendous need for improving the treatment regimens to alleviate the strain to both patient and medical providers and to improve patient outcomes and lower healthcare costs.

In addition to deep burns, skin grafts are used to replace skin in such situations as a large, open wound, a skin infection, a skin ulcer such as a bedsore, or as required from skin cancer surgery.

Skin grafts, and in particular those needed for larger total body surface area burns, may not heal as rapidly and thus result in scar formation. With limitations in the amount of donor skin, for large body surface areas, large mesh ratios are employed. Methods to improve the functional and cosmetic outcomes of skin grafts and in particular those with large mesh ratios, for burns and other reasons, are needed.

The disclosures of all publications, patents, patent application publications and books referred to in this application are hereby incorporated by reference in their entirety into the subject application to more fully describe the art to which the subject disclosure pertains.

### SUMMARY

A method of accelerating or improving the healing of a skin graft or skin grafting site in a subject is provided comprising administering to the subject an amount of an inhibitor of fidgetin-like 2 effective to accelerate healing of the skin graft or skin grafting site. In one embodiment, remodeling of the skin graft is improved. In one embodiment the cosmetic appearance of the skin graft is improved. In one embodiment the skin graft or skin grafting site exhibits a reduced scar formation.

In one embodiment, the inhibitor of fidgetin-like 2 is an siRNA or a shRNA directed to fidgetin-like 2. In one embodiment, the inhibitor is topically applied to the skin grafting site.

In one embodiment, the skin grafting is provided to treat a burn. In one embodiment the burn is a partial-thickness burn. In one embodiment the burn is a full-thickness burn. In one embodiment, the skin grafting is provided to treat an injury, such as from a large open wound. In one embodiment, the skin grafting is provided to treat an ulcer such as but no limited to a bedsore. In one embodiment, the skin grafting is provided to treat a skin infection. In one embodiment, the skin grafting is provided to treat a skin cancer surgery site. In one embodiment, the skin grafting is provided to cover a larger surface area than available from the supply of donor skin.

In one embodiment, the skin graft is comprises a larger total body area burn. In one embodiment, the skin graft has a large meshing ratio. In one embodiment the meshing ratio is greater than 1.5 to 1. In one embodiment the meshing ratio is 3:1. In one embodiment the meshing ratio is 9:1. In one embodiment, the skin graft is a split-thickness skin graft. In one embodiment the skin graft is a full-thickness skin graft.

### BRIEF DESCRIPTION OF THE DRAWINGS

FIG. 1 depicts the porcine burn model.

FIG. 2 shows images of the healing of skin grafts after application of different amounts of FL2-targeted siRNA.

### DETAILED DESCRIPTION

The present subject matter may be understood more readily by reference to the following detailed description which forms a part of this disclosure. It is to be understood that this disclosure is not limited to the specific products, methods, conditions or parameters described and/or shown herein, and that the terminology used herein is for the purpose of describing particular embodiments by way of example only and is not intended to be limiting of the disclosure.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings:

In the present disclosure, the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth.

Similarly, when values are expressed as approximations, by use of the antecedent “about,” it is understood that the particular value forms another embodiment. All ranges are inclusive and combinable. In the context of the present disclosure, by “about” a certain amount it is meant that the amount is within ±20% of the stated amount, or preferably within ±10% of the stated amount, or more preferably within ±5% of the stated amount.

As used herein, the terms “treat”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

The terms “subject,” “individual,” and “patient” are used interchangeably herein, and refer to an animal, for example a human, to whom treatment with a composition or formulation in accordance with the present disclosure, is provided. The term “subject” as used herein refers to human and non-human animals. The human can be any human of any age. In an embodiment, the human is an adult. In another embodiment, the human is a child. The human can be male, female, pregnant, middle-aged, adolescent, or elderly.

A method is provided for accelerating or improving the healing of a skin graft or skin grafting site in a subject comprising administering to the subject an amount of an inhibitor of fidgetin-like 2 effective to accelerate healing of the skin graft or skin grafting site.

Skin grafting sites include those to help recovery from a large, open wound, a skin infection, a burn, an ulcer such as a bedsore, or as required from skin cancer surgery, by way of non-limiting examples. A skin graft may be split-thickness skin graft or a full thickness skin graft.

In certain instances, skin grafting is used to help in the recovery from a large burn. The severity of the burn injury depends on both the total body surface area (TBSA) of the burn as well as the burn depth. The location, depth and general appearance of the burn are used to determine the optimal treatment to improve healing outcomes. Burn depth is typically classified as superficial, partial-thickness, or full-thickness and can serve as a predictor of morbidity and mortality. Superficial burns involve damage to the epidermal layer, which regenerates quickly, and do not result in blisters. Partial-thickness burns consist of damage to both the epidermal and dermal layers. Depending on the depth of damage (papillary versus reticular dermis), partial-thickness burns can heal but this may take an extended period of time and could result in scarring and contracture. Deep partial- and full-thickness burns are characterized by damage to deep dermal and hypodermis. If left untreated, full-thickness burns will likely either not heal or if they do, take several months to heal with the formation of extensive scarring and function-limiting contractures.

Standard of care for deep partial- and full-thickness burns is to surgically excise the eschar followed by early coverage with autologous skin grafts. Skin grafts are meshed to reduce hematoma or seroma formation, allow for wound exudate to pass, and increase the surface area covered by the autograft. The clinical gold standard is a mesh ratio of 1.5:1 split thickness skin graft (STSG) with a thickness determined by the reconstructive needs; although, the STSG do not include adnexal structures. The ratio used, up to 9:1, is dependent on the TBSA required to be treated with a meshed STSG (mSTSG). As TBSA increases, donor site availability decreases resulting in the inability to perform a single-stage autograft. To increase the coverage area, Surgeons have adapted to use either a larger mesh ratio (3:1 or above) or alternative treatments such as allografts and skin substitutes. Unfortunately, both of these options are suboptimal. First, increasing mesh ratio results in delayed reepithelialization and mesh-patterned scarring. For patients with large burn TBSA, improving the functional and cosmetic outcomes of large mesh ratios would reduce the need for two-stage treatments. Second, due to the poor rate of revascularization and cell expansion into skin substitutes, they are limited in clinical feasibility as a single-stage treatment.

The methods and compositions of the disclosure improve the healing of a skin graft for any of the purposes described herein, among any other uses of skin grafting to treat, repair, enhance, or benefit a patient (or mammalian subject) receiving a skin graft. Skin grafts may be derived from the same patient (autologous) or may be from another donor.

In one embodiment, remodeling of the skin graft is improved. In one embodiment the cosmetic appearance of the skin graft is improved. In one embodiment the skin graft or skin grafting site exhibits a reduced scar formation.

In one embodiment, the skin graft is comprises a larger total body area burn. In one embodiment, the skin graft has a large meshing ratio. In one embodiment the meshing ratio is greater than 1.5 to 1. In one embodiment the meshing ratio is 3:1. In one embodiment the meshing ratio is 9:1. In one embodiment, the skin graft is a split-thickness skin graft. In one embodiment the skin graft is a full-thickness skin graft.

In one embodiment, the inhibitor of fidgetin-like 2 is an siRNA or a shRNA directed to fidgetin-like 2. In one embodiment, the inhibitor is topically applied to the skin grafting site.

A method of treating a skin graft or skin grafting site in a subject is provided comprising administering to the subject an amount of an inhibitor of fidgetin-like 2 effective to treat the skin graft or skin grafting site.

In an embodiment, the inhibitor of fidgetin-like 2 is administered locally to the skin graft or skin grafting site. In an embodiment, the inhibitor of fidgetin-like 2 is administered via a vein or artery. In an embodiment, the inhibitor of fidgetin-like 2 is administered by injection, catheterization or cannulation. In an embodiment, the inhibitor of fidgetin-like 2 is administered from an implant that elutes the inhibitor, for example an eluting stent or an eluting skin patch.

In an embodiment, the inhibitor of fidgetin-like 2 is a nucleic acid. In an embodiment, the inhibitor of fidgetin-like 2 is an siRNA or shRNA. In an embodiment, the nucleic acid is directed against a DNA encoding fidgetin-like 2 or against an mRNA encoding fidgetin-like 2.

In an embodiment of the method, the inhibitor of fidgetin-like 2 is encapsulated in a nanoparticle. In an embodiment the nanoparticle is a liposomal nanoparticle.

In an embodiment, the fidgetin-like 2 is human fidgetin-like 2.

In an embodiment, the fidgetin-like 2 comprises consecutive amino acid residues having the sequence set forth in SEQ ID NO:20.

A pharmaceutical composition for treating a skin graft or skin grafting site is provided comprising an amount of an inhibitor of fidgetin-like 2. In an embodiment, the pharmaceutical composition comprises an amount of an inhibitor of fidgetin-like 2 effective to treat a skin graft or skin grafting site in a human subject.

In an embodiment, the pharmaceutical composition comprises a pharmaceutically acceptable carrier.

In an embodiment of the pharmaceutical composition the inhibitor of fidgetin-like 2 is a nucleic acid.

In an embodiment of the pharmaceutical composition the inhibitor of fidgetin-like 2 is an siRNA or shRNA.

In an embodiment of the pharmaceutical composition the nucleic acid is directed against a DNA encoding fidgetin-like 2 or against an mRNA encoding fidgetin-like 2.

In an embodiment of the pharmaceutical composition, the inhibitor of fidgetin-like 2 is encapsulated in a nanoparticle. In an embodiment the nanoparticle is a liposomal nanoparticle.

In an embodiment of the pharmaceutical composition the fidgetin-like 2 1 s human fidgetin-like 2.

In an embodiment of the pharmaceutical composition the fidgetin-like 2 comprises any one of SEQ ID NO:1-18, 23 or 34-72, or any combination thereof. In some embodiments, a double-stranded nucleic acid comprising two sequences from among SEQ ID NOs:1-18 and 34-72 are provided. In some embodiments the pharmaceutical composition comprises more than one single stranded or double stranded nucleic acid.

The dosage of the inhibitor administered in treatment will vary depending upon factors such as the pharmacodynamic characteristics of a specific inhibitor and its mode and route of administration; the age, sex, metabolic rate, absorptive efficiency, health and weight of the recipient; the nature and extent of the symptoms; the kind of concurrent treatment being administered; the frequency of treatment with the inhibitor and the desired therapeutic effect.

A dosage unit of the inhibitor may comprise a single compound, or a mixture of the compound with one or more anti-infection compound(s) or wound healing-promoting compound(s).

In an embodiment, the siRNA (small interfering RNA) as used in the methods or compositions described herein comprises a portion which is complementary to an mRNA sequence encoding a fidgetin-like 2 protein. In an embodiment, the fidgetin-like 2 protein is a human fidgetin-like 2 protein. In an embodiment, the mRNA is encoded by the DNA sequence NCBI Reference Sequence: NM_001013690.4 (SEQ ID NO:19), and the siRNA is effective to inhibit expression of fidgetin-like 2 protein. In an embodiment, the fidgetin-like 2 protein comprises consecutive amino acid residues having the sequence set forth in SEQ ID NO:20.

In an embodiment, the siRNA comprises a double-stranded portion (duplex). In an embodiment, the siRNA is 20-25 nucleotides in length. In an embodiment the siRNA comprises a 19-21 core RNA duplex with a one or two nucleotide 3′ overhang on, independently, either one or both strands. The siRNA can be 5′ phosphorylated, or not, and may be modified with any of the known modifications in the art to improve efficacy and/or resistance to nuclease degradation. In an embodiment the siRNA can be administered such that it is transfected into one or more cells. In an embodiment, the siRNA is 5′ phosphorylated.

In an embodiment, the 5′ terminal residue of a strand of the siRNA is phosphorylated. In an embodiment the 5′ terminal residue of the antisense strand of the siRNA is phosphorylated. In one embodiment, a siRNA of the disclosure comprises a double-stranded RNA wherein one strand of the double-stranded RNA is 80, 85, 90, 95 or 100% complementary to a portion of an RNA transcript of a gene encoding fidgetin-like 2 protein. In an embodiment, the RNA transcript of a gene encoding fidgetin-like 2 protein is an mRNA. In an embodiment, the fidgetin-like 2 protein is a human fidgetin-like 2 protein. In an embodiment, a siRNA of the disclosure comprises a double-stranded RNA wherein one strand of the RNA comprises a portion having a sequence the same as a portion of 18-25 consecutive nucleotides of an RNA transcript of a gene encoding fidgetin-like 2 protein. In an embodiment, the fidgetin-like 2 protein is a human fidgetin-like 2 protein. In yet another embodiment, a siRNA of the disclosure comprises a double-stranded RNA wherein both strands of RNA are connected by a non-nucleotide linker. Alternately, a siRNA of the disclosure comprises a double-stranded RNA wherein both strands of RNA are connected by a nucleotide linker, such as a loop or stem loop structure.

In one embodiment, a single strand component of a siRNA of the disclosure is from 14 to 50 nucleotides in length. In another embodiment, a single strand component of a siRNA of the disclosure is 14, 15, 16, 17, 18, 19, 20, 21, 22, 23, 24, 25, 26, 27, or 28 nucleotides in length. In yet another embodiment, a single strand component of a siRNA of the disclosure is 21 nucleotides in length. In yet another embodiment, a single strand component of a siRNA of the disclosure is 22 nucleotides in length. In yet another embodiment, a single strand component of a siRNA of the disclosure is 23 nucleotides in length. In one embodiment, a siRNA of the disclosure is from 28 to 56 nucleotides in length. In another embodiment, a siRNA of the disclosure is 40, 41, 42, 43, 44, 45, 46, 47, 48, 49, 50, 51, or 52 nucleotides in length.

In another embodiment, an siRNA of the disclosure comprises at least one 2′-sugar modification. In another embodiment, an siRNA of the disclosure comprises at least one nucleic acid base modification. In another embodiment, an siRNA of the disclosure comprises at least one phosphate backbone modification. As used herein, “at least one” means one or more.

In one embodiment, RNAi inhibition of fidgetin-like 2 protein is effected by a short hairpin RNA (“shRNA”). The shRNA is introduced into the appropriate cell by transduction with a vector. In an embodiment, the vector is a lentiviral vector. In an embodiment, the vector comprises a promoter. In an embodiment, the promoter is a U6 or Hl promoter. In an embodiment the shRNA encoded by the vector is a first nucleotide sequence ranging from 19-29 nucleotides complementary to the target gene/mRNA, in the present case the mRNA encodes fidgetin-like 2 protein. In an embodiment the fidgetin-like 2 protein is a human fidgetin-like 2 protein. In an embodiment the shRNA encoded by the vector also comprises a short spacer of 4-15 nucleotides (a loop, which does not hybridize) and a 19-29 nucleotide sequence that is a reverse complement of the first nucleotide sequence. In an embodiment the siRNA resulting from intracellular processing of the shRNA has overhangs of 1 or 2 nucleotides. In an embodiment the siRNA resulting from intracellular processing of the shRNA overhangs has two 3′ overhangs. In an embodiment the overhangs are UU.

In one embodiment a shRNA to FL2 useful for the purposed disclosed herein comprises the sequence CACCGCTGGAGCCCTTTGACAAGTTCTCGAGAACTTGTCAAAGGGCTCCAGCTT TT (SEQ ID NO:23). In one embodiment a shRNA to FL2 consists of the sequence CACCGCTGGAGCCCTTTGACAAGTTCTCGAGAACTTGTCAAAGGGCTCCAGCTT TT (SEQ ID NO:23).

NCBI Reference Sequence: NM_001013690.4 (SEQ ID NO:19) (nucleic acid encoding human fidgetin-like 2):

| | |
|---|---|
| 1 | agtgagctat ggggacacta ctgcactgta gcctgggcaa |
| | cagagcaaga ccttgtctca |
| 61 | aaaatgtata tatattttgg gctttttttc ctaaaacggg |
| | aactacaaca gcatatttgc |
| 121 | gagctgatga gagtgaccca gcagagaggg aaatggatca |
| | gctctgttga agatgcactg |
| 181 | gacaccagaa cacgcccagc ccctcaacca gtggccagag |
| | cagcacctgg acgtctcctc |
| 241 | caccaccccg tcgccggccc acaagttgga gttgccccct |
| | gggggtcgcc aacgctgcca |
| 301 | ctacgcttgg gcacacgacg acatctcagc cctcactgcc |
| | tccaacctcc taaagcgcta |
| 361 | tgcagagaag tactctgggg tcttggattc tccctacgag |
| | cgtccggccc tgggcgggta |
| 421 | cagcgacgcc tccttcctca acggcgccaa aggggatccc |
| | gagccctggc cagggccgga |
| 481 | gccaccctac cccttggcct cactccacga aggcctccca |
| | ggaaccaaat cgggcggtgg |
| 541 | cggcggttcc ggggccctgg ggggctcccc agttttagcc |
| | gggaacctcc ctgaacccct |
| 601 | ctacgccggc aatgcgtgcg ggggcccatc ggcggcgccc |
| | gagtacgcgg ccggctacgg |
| 661 | cggggggtac ctggcgccgg gttactgcgc gcagacgggc |
| | gccgcgctgc ccccgccgcc |
| 721 | cccggccgcg ctcctgcagc ccccaccgcc tccggggtac |
| | gggccctcag cgccgctgta |
| 781 | caactatccc gcagggggct acgcagcgca gcccggctat |
| | ggcgcgctcc cgccgccccc |
| 841 | aggcccaccc ccggccccct acctgacccc gggcctgccc |
| | gcgcccacgc ccctgcccgc |
| 901 | gccggcaccg cccaccgcct atggcttccc cacggccgcg |
| | ccgggtgccg aatccgggct |
| 961 | gtcgctgaag cgcaaggccg ccgacgaggg gcccgagggc |
| | cgctaccgca agtacgcgta |
| 1021 | cgagcccgcc aaggcccccg tggctgacgg agcctcctac |
| | cccgccgcgg acaacggcga |
| 1081 | atgtcggggc aacgggttcc gggccaagcc gccaggagcc |
| | gcggaggagg cgtcgggcaa |
| 1141 | gtacggtggc ggcgtccccc tcaaggtcct gggctccccc |
| | gtctacggcc cgcaactgga |
| 1201 | gccctttgaa aagttcccgg agcgggcccc ggctcctcgt |
| | ggggggttcg ccgtgccgtc |
| 1261 | gggggagact cccaaaggcg tggaccctgg ggccctggag |
| | ctggtgacga gcaagatggt |
| 1321 | ggactgcggg cccccggtgc agtgggcgga tgtggcgggc |
| | cagggcgcgc tcaaggcggc |
| 1381 | gctggaggag gagctggtgt ggcccctgct caggccgccc |
| | gcctacccgg gcagcctgcg |
| 1441 | cccgccgcgg accgtcctgc tctttgggcc gcggggcgcg |
| | ggcaaagcgc tgctgggccg |
| 1501 | ctgcctcgcc acgcagctgg gcgccacgct gttgcgcctg |
| | cgcggcgcga ccctggctgc |
| 1561 | gcccggcgcc gccgagggcg cgcgcctcct ccaggccgcc |
| | ttcgcggccg cgcgctgccg |
| 1621 | cccaccctcc gtactcctca tcagcgagct agaggcgctg |
| | ctccccgccc gggacgacgg |
| 1681 | cgcggcggca gggggcgcgc tgcaggtgcc gctcctggcc |
| | tgcctggacg ggggctgcgg |
| 1741 | cgcgggggct gacggcgtgc tggttgtggg caccacctcg |
| | cggcccgcgg ctctggacga |
| 1801 | ggcgacccgc cggcgcttct ctctccgctt ctacgtggcg |
| | ctgcccgaca gcccggcccg |
| 1861 | cgggcagatc ctgcagcggg cgctggccca gcagggctgc |
| | gcgctcagtg agcgggaact |
| 1921 | ggcggcgctg gtgcagggca cgcagggctt ctctgggggc |
| | gagctggggc agctgtgcca |
| 1981 | gcaggcggcg gccggggcgg gcctcccggg gctgcagcgc |
| | cccctctcct acaaggacct |
| 2041 | ggaggcggcg ctggccaagg tgggccctag ggcctctgcc |
| | aaggaactgg actcgttcgt |
| 2101 | ggagtgggac aaaatgtacg gctccggaca ctgacggcgc |
| | gcgggggagg ccgcgggagc |
| 2161 | cgcagtccct ccgtccccgc cgcctccgcg tgggagggat |
| | gtcactgact aaacccggct |
| 2221 | ggcaggggct ggagtggtga atgtgggatc ggggacagga |
| | ggggtctgcc ggtggatatt |
| 2281 | ttttttttcg tgggaaggaa aatgcttctg ccaggcagat |
| | gccatatgcg ccgtgtactc |
| 2341 | aggtttttcc tatttattgt ggactggaag ctcgccatct |
| | ccgcccggca gaccgggcag |
| 2401 | atccggcatg ggctggcacc cggggcctta agaactcctg |
| | ctctcttgcc acaacgcttt |
| 2461 | tgtctcctcg ctatctgaat ggcaccctcc ttctccctca |
| | ctctctccat cccattctct |
| 2521 | gcattctctt ggttttctct cccttttgct ttgtcgctga |
| | cacccctgcc caccccatgc |
| 2581 | tggccctgtt tctctcctgc ccctccctcc ccagctctcc |
| | atccctcacc ctctgtgctt |
| 2641 | ctgtctccat ccctggctct ccagcgtccc tggccttttg |
| | gtccctgagc tttaatgcct |
| 2701 | ttccctgcct tctgttctta tttggactgc agtggccctt |
| | tgcaggagct ctggaggccc |
| 2761 | aggggctgag gaggagggtt acccctctac ccatctgaaa |
| | cctagggtct agggggatca |
| 2821 | aggaaaaaaa gtccccaaag aaggggaatt ttttgtttgt |
| | ttttgagggg agatcccaga |
| 2881 | aatgtagctt gtttcatatt ttagtcttct tatttttgta |
| | aaatgtgtag aatttgctgt |
| 2941 | ttttcttttt cttttgacaa ctcaggaaga aactgacctc |
| | agaaagaatg ttagactttg |
| 3001 | gctgctctcc tgtgtgcccc tcacacctgc cccctccccc |
| | ccactccatc caggggacca |
| 3061 | aattctccca gacactcaaa aaatgagact tacggggaag |
| | gggagaggaa gacccagagg |
| 3121 | cctcagtgaa accccagcta ttcctggtca gaagcagaat |
| | gtattcctaa gggcttcctc |
| 3181 | cccagggccg aggcctaggc atgaatgtgg ggagtgggct |
| | gtggggtttg agagaaggga |
| 3241 | ggccttattc ctctcctgct gctccccacc ccctgcccca |
| | cccaacccct ccgctgagtg |
| 3301 | ttttctgtga agggctatcc agagttagga tgcccttgcc |
| | caattccttc ctgagaccca |
| 3361 | gaaggtaggg tgggagggcc caaatgggaa ggtgacctaa |
| | gcagaaagtc tccagaaagg |
| 3421 | tcatgtcccc tggccctgcc ttggcagagg tccccagtga |
| | cttatgctag gaggattcca |
| 3481 | tctgggtaga cagtctggcc acaaaatcag ctactggacc |
| | tcagccatct ctgctggagg |
| 3541 | ctctgaggag gagtgagcat ccctcacttg tgggggctct |
| | gtgaggaaat gtgccttccc |
| 3601 | cattcccccg gagtcctagg tctggagctc cagggctggg |
| | agagggtgag ggagatgggc |
| 3661 | aggggtgttt tctctgacct tgggggctta gtctcagtcc |
| | tgcctgaact ttccactagg |
| 3721 | cttggaaccc ttccaagaac catatttctc tccttcccac |
| | caattttccc ttgatgaggc |
| 3781 | tttagcagtt tgctcccacc acccccagcc catttcacaa |
| | ctctgatctt agtccaaagc |
| 3841 | aggggacacg cccccccacc accacttttt ctctctccca |
| | tctcagcctc ctgtgcagtt |
| 3901 | ccttgcctgc ccgtgcattt cctagagtct actgcctccc |
| | ccctggctgg gagggtgtct |
| 3961 | gggggggatc tttcaggggc cctggcaccc agggcctgtg |
| | ctggcctagg agtgctgacc |
| 4021 | agaaggctgc tctgttcccc cccacccccg ttgctttctg |
| | gccccctctt tggagccagc |
| 4081 | cacccacagg gctttggtgc ctcagaagca gtgggctgcc |
| | gggtcacagc cgcaggctgc |
| 4141 | aaaagaccct cggagggagc atggagtgag gggttctctc |
| | tcaggtgtgt atgtattggg |
| 4201 | gggtgggggt gggtggaggg tgtcagggaa gttggggtgg |
| | gatcccagcc ttcccttcaa |
| 4261 | gaggcaggga gctctgggag gtggagtccc caccgctttc |
| | tctactaggc tcctcctgtt |
| 4321 | ccccaggctt ggggagcttt gcacaaggag actgccccca |
| | gcctagtggc acctacctca |
| 4381 | tgggctctgg ggcaggtagg ggaagggcca gtccagctct |
| | ggtaatgctg gggggaggca |
| 4441 | taccaaagaa tccaggggca gggagtgggg agggtgactt |
| | ccgagctggc ctctcccctt |
| 4501 | cctctaccca gactggggct gggatcctct cctcccgctg |
| | taaccatttc tacctcattt |
| 4561 | tgctgcgtgt tgtacatgga cgtatttatc tcctgtctga |
| | cgatgctctg cagttgtggt |
| 4621 | ctgtctacct cagaagagac tgtattttaa aagaaagtat |
| | tacacagtat taaagcgatg |
| 4681 | acatgtggtt tgcaaaaaaa aaaaaaaaaa a |

which encodes:

(SEQ ID NO: 20)
MHWTPEHAQPLNQWPEQHLDVSSTTPSPAHKLELPPGGRQRCHYAWAHDD
ISALTASNLLKRYAEKYSGVLDSPYERPALGGYSDASFLNGAKGDPEPWP
GPEPPYPLASLHEGLPGTKSGGGGGSGALGGSPVLAGNLPEPLYAGNACG
GPSAAPEYAAGYGGGYLAPGYCAQTGAALPPPPPAALLQPPPPPGYGPSA
PLYNYPAGGYAAQPGYGALPPPPGPPPAPYLTPGLPAPTPLPAPAPPTAY
GFPTAAPGAESGLSLKRKAADEGPEGRYRKYAYEPAKAPVADGASYPAAD
NGECRGNGFRAKPPGAAEEASGKYGGGVPLKVLGSPVYGPQLEPFEKFPE
RAPAPRGGFAVPSGETPKGVDPGALELVTSKMVDCGPPVQWADVAGQGAL
KAALEEELVWPLLRPPAYPGSLRPPRTVLLFGPRGAGKALLGRCLATQLG
ATLLRLRGATLAAPGAAEGARLLQAAFAAARCRPPSVLLISELEALLPAR
DDGAAAGGALQVPLLACLDGGCGAGADGVLVVGTTSRPAALDEATRRRFS
LRFYVALPDSPARGQILQRALAQQGCALSERELAALVQGTQGFSGGELGQ
LCQQAAAGAGLPGLQRPLSYKDLEAALAKVGPRASAKELDSFVEWDKMYG
SGH (human fidgetin-like 2).

In various embodiments herein, siRNAs useful for the purposes disclosed herein are described as follows. In embodiments, the siRNA useful for the purposes disclosed herein comprise one of the following pairs of sense/antisense sequences:

| | Sense: |
|---|---|
| (SEQ ID NO: 1) | |
| | 5′ UUACACAGUAUUAAAGCGAUU |
| | and |
| | Antisense: |
| (SEQ ID NO: 2) | |
| | 5′ UCGCUUUAAUACUGUGUAAUU; |
| | Sense: |
| (SEQ ID NO: 3) | |
| | 5′ CAUCUGAAACCUAGGGUCUUU |
| | and |
| | Antisense: |
| (SEQ ID NO: 4) | |
| | 5′ AGACCCUAGGUUUCAGAUGUU; |
| | Sense: |
| (SEQ ID NO: 5) | |
| | 5′ GUGACUUAUGCUAGGAGGAUU |
| | and |
| | Antisense: |
| (SEQ ID NO: 6) | |
| | 5′ UCCUCCUAGCAUAAGUCACUU; |
| | Sense: |
| (SEQ ID NO: 7) | |
| | 5′ GGUCAGAAGCAGAAUGUAUUU |
| | and |
| | Antisense: |
| (SEQ ID NO: 8) | |
| | 5′ AUACAUUCUGCUUCUGACCUU; |
| | or |
| | Sense: |
| (SEQ ID NO: 9) | |
| | 5′ CGCCGGCCCACAAGUUGGAdTdT |
| | and |
| | Antisense: |
| (SEQ ID NO: 10) | |
| | 5′ UCCAACUUGUGGGCCGGCGdTdT; |
| | Sense: |
| (SEQ ID NO: 11) | |
| | 5′ CAGCUCGAGCCCUUUGACAdTdT |
| | and |
| | Antisense: |
| (SEQ ID NO: 12) | |
| | 5′ UGUCAAAGGGCUCGAGCUGdTdT; |
| | Sense: |
| (SEQ ID NO: 13) | |
| | 5′ CCUCCAACCUCCUCAAGAGdTdT |
| | and |
| | Antisense: |
| (SEQ ID NO: 14) | |
| | 5′ CUCUUGAGGAGGUUGGAGGdTdT; |
| | or |
| | Sense: |
| (SEQ ID NO: 15) | |
| | 5′ CGUUGCUGCUCAUCAGCGAdTdT |
| | and |
| | Antisense: |
| (SEQ ID NO: 16) | |
| | 5′ UCGCUGAUGAGCAGCAACGdTdT. |

In some embodiments, a pharmaceutical composition for the uses as described herein may comprise any one or more of the foregoing single-stranded siRNA sequences. In some embodiments, a pharmaceutical composition for the uses as described herein may comprise any one or more of the foregoing single-stranded siRNA sequences in a duplex with another single-stranded sequence, selected from any of those disclosed herein, or selected from any other sequence.

In some embodiments, the siRNA useful for the purposes disclosed herein consists of one of the following sequences:

| | Sense strand: |
|---|---|
| (SEQ ID NO: 17) | |
| | 5′ - fUfUmAfCmAfCAGUAUUAAAGCGATT; |
| | Antisense strand: |
| (SEQ ID NO: 18) | |
| | (Phos) 5′ - U CGC UUU AAU ACU G UG UAA TT; |
| | Sense strand: |
| (SEQ ID NO: 34) | |
| | 5′ - UUACACAGUAUUAAAGCGATT - 3′; |
| | Antisense strand: |
| (SEQ ID NO: 35) | |
| | (Phos) 5′ - mUmCGCUUUAAUACUGUGUAATT - 3′; |
| | Antisense strand: |
| (SEQ ID NO: 36) | |
| | (Phos) 5′ - mU(s)mC(s)GCUUUAAUACUGUGUAATT -3′; |
| | Antisense strand: |
| (SEQ ID NO: 37) | |
| | (Phos) 5′ - fUfCGCUUUAAUACUGUGUAATT - 3′; |
| | Antisense strand: |
| (SEQ ID NO: 38) | |
| | (Phos) 5′ -fU(s)fC(s)GCUUUAAUACUGUGUAATT - 3′; |
| | Antisense strand: |
| (SEQ ID NO: 39) | |
| | (Phos) 5′ - mU(s)mC(s)GCUUUAAUAmCfUmGfUmGfUmAm |
| | ATT - 3′; |
| | Antisense strand: |
| (SEQ ID NO: 40) | |
| | (Phos) 5′ - U(s)CGCUUUAAUACUGUGUAATT - 3′; |
| | Antisense strand: |
| (SEQ ID NO: 41) | |
| | (Phos) 5′ - mUfCmGfCmUfUmUAAfUmAfCmUGmUmGfUmAm |
| | ATT - 3′; |
| | Sense strand: |
| (SEQ ID NO: 42) | |
| | 5′ - mUmUmAmCmAmCmAmGmUmAmUmUmAmAmAmGmCmGmAmUm |
| | U - 3′; |
| | Antisense strand: |
| (SEQ ID NO: 43) | |
| | (Phos) 5′ - mUmCmGmCmUmUmUmAmAmUmAmCmUmGmUmGmU |
| | mAmAmUmU - 3′; |
| | Sense strand: |
| (SEQ ID NO: 44) | |
| | 5′ - mUmUmAmCmAmCmAmGmUmAmUmUmAmAmAmGdCdGdAT |
| | T - 3′; |
| | Sense strand: |
| (SEQ ID NO: 45) | |
| | 5′ - mUmUmAmCmAmCmAmGmUmAmUmUmAmAmAmGdCmGmAT |
| | T - 3′; |
| | Sense strand: |
| (SEQ ID NO: 46) | |
| | 5′ - UUACACAGUAUUAAAGCGA - 3′; |
| | Antisense strand: |
| (SEQ ID NO: 47) | |
| | (Phos) 5′ - U(s)CGCUUUAAUACUGUGUAATT - 3′; |
| | Antisense strand: |
| (SEQ ID NO: 48) | |
| | (Phos) 5′ - UCGCUUUAAUACUGUGUAATT - 3′; |
| | Antisense strand: |
| (SEQ ID NO: 49) | |
| | (Phos) 5′ - U(s)C(s)GCUUUAAUACUGUGUAATT - 3′; |
| | Sense strand: |
| (SEQ ID NO: 50) | |
| | 5′ - mUmUACACAGUAUUAAAGCGA - 3′; |
| | Antisense strand: |
| (SEQ ID NO: 51) | |
| | (Phos) 5′ - U(s)CGCUUUAAUACUGUGUmAmATT - 3′; |
| | Antisense strand: |
| (SEQ ID NO: 52) | |
| | (Phos) 5′ - UCGCUUUAAUACUGUGUAATT - 3′; |
| | Antisense strand: |
| (SEQ ID NO: 53) | |
| | (Phos) 5′ - U(s)C(s)GCUUUAAUACUGUGUAA T(s)T - |
| | 3′; |
| | Sense strand: |
| (SEQ ID NO: 54) | |
| | 5′ - lUlUlAlClACAGUAUUAAAGCGATT - 3′; |
| | Antisense strand: |
| (SEQ ID NO: 55) | |
| | (Phos) 5′ - UCGCUUUAAUACUGlUlGlUlAlA TT - 3′; |
| | Sense strand: |
| (SEQ ID NO: 56) | |
| | 5′ - fUfUlAfClACAGUAUUAAAGCGA - 3′ |
| | Antisense strand: |
| (SEQ ID NO: 57) | |
| | (Phos) 5′ - mU(s)mCmGCUUUAAUACUGUGUAATT - 3′; |
| | Antisense strand: |
| (SEQ ID NO: 58) | |
| | 5′ - fUfCGCUUUAAUACUGUGUAATT - 3′; |
| | Antisense strand: |
| (SEQ ID NO: 59) | |
| | 5′ -fU(s)fC(s)GCUUUAAUACUGUGUAATT - 3′; |
| | Antisense strand: |
| (SEQ ID NO: 60) | |
| | 5′ - mU(s)mC(s)GCUUUAAUAmCfUmGfUmGfUmAmATT - |
| | 3′; |
| | Antisense strand: |
| (SEQ ID NO: 61) | |
| | 5′ - U(s)CGCUUUAAUACUGUGUAATT - 3′; |
| | Antisense strand: |
| (SEQ ID NO: 62) | |
| | 5′ - mUfCmGfCmUfUmUAAfUmAfCmUGmUmGfUmAmATT; |
| | Antisense strand: |
| (SEQ ID NO: 63) | |
| | 5′ - mUmCmGmCmUmUmUmAmAmUmAmCmUmGmUmGmUmAmAmUm |
| | U - 3′; |
| | Antisense strand: |
| (SEQ ID NO: 64) | |
| | 5′ - U(s)CGCUUUAAUACUGUGUAATT - 3′; |
| | Antisense strand: |
| (SEQ ID NO: 65) | |
| | 5′ - UCGCUUUAAUACUGUGUAATT - 3′; |
| | Antisense strand: |
| (SEQ ID NO: 66) | |
| | 5′ - U(s)C(s)GCUUUAAUACUGUGUAATT - 3′; |
| | Antisense strand: |
| (SEQ ID NO: 67) | |
| | 5′ - U(s)CGCUUUAAUACUGUGUmAmATT - 3′; |
| | Antisense strand: |
| (SEQ ID NO: 68) | |
| | 5′ - UCGCUUUAAUACUGUGUAATT - 3′; |
| | Antisense strand: |
| (SEQ ID NO: 69) | |
| | 5′ - U(s)C(s)GCUUUAAUACUGUGUAA T(s)T - 3′; |
| | Antisense strand: |
| (SEQ ID NO: 70) | |
| | 5′ - UCGCUUUAAUACUGlUlGlUlAlA TT - 3′; |
| | Antisense strand: |
| (SEQ ID NO: 71) | |
| | 5′ - mU(s)mCmGCUUUAAUACUGUGUAATT - 3′; |
| | or |
| | Antisense strand: |
| (SEQ ID NO: 72) | |
| | 5′ - U CGC UUU AAU ACU G UG UAA TT; |

and a complement;
wherein d(nucleotide)=deoxy-(nucleotide), m(nucleotide)=2′-O-methyl nucleotide, T=thymidine, f(nucleotide)=2′-fluorodeoxy nucleotide, (Phos)=phosphodiester cap; capital letter nucleotide=RNA nucleotide, l(nucleotide)=a locked nucleotide, and (s)=phosphorothioate. Thus, for example dT represents deoxythymidine, dC represents deoxycytidine, fC represents 2′-fluorodeoxy cytidine ribonucleic acid, fU represents 2′-fluorodeoxy uracil ribonucleic acid, mA represents 2′-O-methyl adenosine ribonucleic acid, mU represents 2′-O-methyl uracil ribonucleic acid, mC represents 2′-O-methyl cytosine ribonucleic acid, and mG represents 2′-O-methyl guanosine ribonucleic acid.

In some embodiments, siRNA useful for the purposes disclosed herein comprises any sequence from among SEQ ID NOs: 1-18 and 34-72. In some embodiments, siRNA useful for the purposes described herein comprises a double-stranded siRNA comprising any sequence among SEQ ID NOs:1-18 and 34-72, and a complementary sequence consisting of any sequence among SEQ ID NOs:1-18 and 34-72.

In some embodiments, the siRNA may have a 5′-phosphodiester cap, as abbreviated “(Phos)” in the aforementioned sequences. In some embodiments, the siRNA does not have a 5′-phosphodiester cap. siRNA sequences without a 5′-phosphodiester cap are fully embraced herein.

A phosphorothioate linkage between nucleotides is represented in the sequences by “(s)”.

Locked nucleotides in one embodiment comprise a ribose with a 2′-O, 4′-C methylene bridge, for example, 2′-O, 4′-C methylene adenosine (lA); 2′-O, 4′-C methylene guanosine (lG); 2′-O, 4′-C methylene cytidine (lC); 2′-O, 4′-C methylene uridine (lU); and 2′-O, 4′-C methylene thymine (lT) ribonucleosides. In other embodiments, the locked nucleic acid comprises a methyl group attached to the methylene group. Other types of locked nucleic acids are embraced herein.

In one embodiment, the FL2 siRNA useful for the purposes disclosed herein is double-stranded and comprises any complementary sense sequence and antisense sequence from the foregoing SEQ ID NOs:1-18 and 34-72. In an embodiment, the siRNA is double-stranded and comprises SEQ ID NO:1 and SEQ ID NO:2; SEQ ID NO:3 and SEQ ID NO:4; SEQ ID NO:5 and SEQ ID NO:6; or SEQ ID NO:7 and SEQ ID NO:8.

In an embodiment, the 5′ terminal residue of a strand of the siRNA is phosphorylated. In an embodiment the 5′ terminal residue of the antisense strand of the siRNA is phosphorylated

As used herein an “aptamer” is a single-stranded oligonucleotide or oligonucleotide analog that binds to a particular target molecule, such as a fidgetin-like 2 protein, or to a nucleic acid encoding a fidgetin-like 2 protein, and inhibits the function or expression thereof, as appropriate. Alternatively, an aptamer may be a protein aptamer which consists of a variable peptide loop attached at both ends to a protein scaffold that interferes with fidgetin-like 2 protein interactions.

In one embodiment, complement refers to the complementary nucleic acid strand comprising a double-stranded nucleic acid. In one embodiment, if a sense strand is selected, its complement is an antisense strand. In one embodiment if an antisense strand is selected, its complement is a sense strand.

In one embodiment, the complement may be selected from any of SEQ ID NO:1-18 and 34-72. In one embodiment, if the siRNA molecule is a sense strand from among SEQ ID NOs: 1-18 and 34-72, the complement may be selected from any antisense strand from among SEQ ID NOs: 1-18 and 34-72. In one embodiment, if the siRNA molecule is an antisense strand from among SEQ ID NOs: 1-18 and 34-72, the complement may be selected from any sense strand from among SEQ ID NOs: 1-18 and 34-72.

In one embodiment, the complement may be selected from SEQ ID NOs:1-16. In one embodiment, if the siRNA molecule is a sense strand from among SEQ ID NOs:1-18 and 34-72, the complement may be selected from an antisense strand from among SEQ ID NOs:1-16. In one embodiment, if the siRNA molecule is an antisense strand from among SEQ ID NOs: 1-18 and 34-72, the complement may be selected from a sense strand from among SEQ ID NOs:1-16.

In some embodiment, any of the nucleic acid sequences disclosed herein may be modified or further modified with one or more nucleotide modifications as described herein. In one embodiment, any unmodified nucleotide in a sequence described herein may be modified to one of the modified nucleotides such as but not limited to those described herein. In one embodiment, a modified nucleotide in a sequence described herein may be changed to a different modified nucleotide such as but not limited to one of the modified nucleotides described herein. Modified nucleotide or modified nucleic acid encompasses modified nucleotides, bonds between nucleotides or any component of a nucleotide, and addition of one or more modified or unmodified nucleotides to one or both ends of a sequence, or addition of a cap, as described herein.

In one embodiment, a double stranded nucleic acid is provided consisting of two nucleic acid molecules selected from among SEQ ID NOs: 1-18 and 34-72.

Non-limiting examples of such double-stranded sequences include SEQ ID NO:1 and SEQ ID NO: 2, SEQ ID NO:3 and SEQ ID NO: 4, SEQ ID NO:5 and SEQ ID NO: 6, SEQ ID NO:7 and SEQ ID NO: 8, SEQ ID NO:9 and SEQ ID NO: 10, SEQ ID NO:11 and SEQ ID NO: 12, SEQ ID NO:13 and SEQ ID NO: 14, SEQ ID NO:15 and SEQ ID NO: 16, and SEQ ID NO:17 and SEQ ID NO: 18. In some embodiments, the siRNA is single-stranded, selected from among SEQ ID NO:1-18 above.

In one embodiment, a double stranded nucleic acid is provided consisting of complementary nucleic acid molecules selected from among SEQ ID NOs: 34-57 or from among SEQ ID NOS: 1-18 or 34-57. In one embodiment, the double stranded nucleic acid comprises a sense strand and an antisense strand. In one embodiment, the double stranded nucleic acid consists of a sense strand and an antisense strand.

In one embodiment, a double stranded nucleic acid is provided consisting of a sense strand selected from SEQ ID NOs: 1, 3, 5, 7, 9, 11, 13, 15 or 17; and an antisense strand selected from SEQ ID NOs: 2, 4, 6, 8, 10, 1, 2, 14, 16 or 18.

In one embodiment, a double stranded nucleic acid is provided consisting of a sense strand selected from SEQ ID NOs: 1, 17, 34, 42, 44, 45, 46, 50 and 54; and an antisense strand selected from SEQ ID NOs: 2, 18, 35, 36, 37, 38, 39, 40, 41, 43, 47, 48, 49, 51, 52, 53, 55 and 57.

In one embodiment, a double stranded nucleic acid is provided consisting of a sense strand selected from SEQ ID NOs: 1, 17, 34, 42, 44, 45, 46, 50, 54 and 56; and an antisense strand selected from SEQ ID NOs: 2, 4, 6, and 8.

In one embodiment, a double stranded nucleic acid is provided consisting of a sense strand selected from SEQ ID NOs: 1, 3, 5 and 7; and an antisense strand selected from SEQ ID NOs: 18, 35, 36, 37, 38, 39, 40, 41, 43, 47, 48, 49, 51, 52, 53, 55 and 57.

In one embodiment, a double stranded nucleic acid is provided consisting of a sense strand selected from SEQ ID NOs: 1, 3, 5, 7, 9, 11, 13, 15, 17, 34, 42, 44, 45, 46, 50 and 54; and an antisense strand selected from SEQ ID NOs: 2, 4, 6, 8, 10, 1, 2, 14, 16, 18, 35, 36, 37, 38, 39, 40, 41, 43, 47, 48, 49, 51, 52, 53, 55 and 57.

In one embodiment, a double stranded nucleic acid is provided comprising a sense strand selected from SEQ ID NOs: 1, 3, 5, 7, 9, 11, 13, 15, 17, 34, 42, 44, 45, 46, 50 and 54; and an antisense strand selected from SEQ ID NOs: 2, 4, 6, 8, 10, 1, 2, 14, 16, 18, 35, 36, 37, 38, 39, 40, 41, 43, 47, 48, 49, 51, 52, 53, 55 and 57.

In one embodiment, a double-stranded nucleic acid is provided consisting of SEQ ID NO:17 and SEQ ID NO:18; SEQ ID NO:34 and SEQ ID NO:35; SEQ ID NO:34 and SEQ ID NO:36; SEQ ID NO:34 and SEQ ID NO:37; SEQ ID NO:34 and SEQ ID NO:38; SEQ ID NO:34 and SEQ ID NO:39; SEQ ID NO:17 and SEQ ID NO:40; SEQ ID NO:34 and SEQ ID NO:41; SEQ ID NO:42 and SEQ ID NO:43; SEQ ID NO:44 and SEQ ID NO:43; SEQ ID NO:45 and SEQ ID NO:43; SEQ ID NO:46 and SEQ ID NO:47; SEQ ID NO:46 and SEQ ID NO:48; SEQ ID NO:46 and SEQ ID NO:49; SEQ ID NO:50 and SEQ ID NO:51; SEQ ID NO:46 and SEQ ID NO:53; SEQ ID NO:54 and SEQ ID NO:55; or SEQ ID NO:56 and SEQ ID NO:57.

In one embodiment, a double stranded nucleic acid is provided comprising at least one nucleic acid molecule selected from among SEQ ID NOs: 1-18 or 34-57.

In one embodiment, a double stranded nucleic acid is provided comprising two nucleic acid molecules selected from among SEQ ID NOs: 1-18 or 34-57. In one embodiment, the double stranded nucleic acid comprises a sense strand and an antisense strand.

In one embodiment, each strand of the double stranded nucleic acid has no more than 52 nucleotides.

In one embodiment, a double stranded nucleic acid is provided comprising a sense strand comprising a nucleic acid molecule selected from SEQ ID NOs: 1, 17, 34, 42, 44, 45, 46, 50, 54 and 56; and an antisense strand comprising a nucleic acid molecule selected from SEQ ID NOs: 2, 18, 35, 36, 37, 38, 39, 40, 41, 43, 47, 48, 49, 51, 52, 53, 55 and 57.

In one embodiment, a double stranded nucleic acid is provided comprising a sense strand comprising a nucleic acid molecule selected from SEQ ID NOs: 1, 17, 34, 42, 44, 45, 46, 50, 54 and 56; and an antisense strand comprising a nucleic acid molecule selected from SEQ ID NOs: 4, 6, 8, and 10.

In one embodiment, a double stranded nucleic acid is provided comprising a sense strand comprising a nucleic acid molecule selected from SEQ ID NOs: 1, 3, 5, 7 and 9; and an antisense strand comprising a nucleic acid molecule selected from SEQ ID NO: 18, 35, 36, 37, 38, 39, 40, 41, 43, 47, 48, 49, 51, 52, 53, 55 and 57.

In one embodiment, the double-stranded nucleic acid comprises nucleic acid molecules comprising SEQ ID NO:17 and SEQ ID NO:18; SEQ ID NO:34 and SEQ ID NO:35; SEQ ID NO:34 and SEQ ID NO:36; SEQ ID NO:34 and SEQ ID NO:37; SEQ ID NO:34 and SEQ ID NO:38; SEQ ID NO:34 and SEQ ID NO:39; SEQ ID NO:17 and SEQ ID NO:40; SEQ ID NO:34 and SEQ ID NO:41; SEQ ID NO:42 and SEQ ID NO:43; SEQ ID NO:44 and SEQ ID NO:43; SEQ ID NO:45 and SEQ ID NO:43; SEQ ID NO:46 and SEQ ID NO:47; SEQ ID NO:46 and SEQ ID NO:48; SEQ ID NO:46 and SEQ ID NO:49; SEQ ID NO:50 and SEQ ID NO:51; SEQ ID NO:46 and SEQ ID NO:53; SEQ ID NO:54 and SEQ ID NO:55; or SEQ ID NO:56 and SEQ ID NO:57.

In one embodiment, a double stranded nucleic acid is provided consisting of a sense strand selected from SEQ ID NOs: 1, 17, 34, 42, 44, 45, 46, 50 and 54; and an antisense strand selected from any one of SEQ ID NOs: 58-72.

In one embodiment, a double stranded nucleic acid is provided consisting of a sense strand selected from SEQ ID NOs: 1, 3, 5 and 7; and an antisense strand selected from any one of SEQ ID NOs: 58-72.

In one embodiment, a double stranded nucleic acid is provided consisting of a sense strand selected from SEQ ID NOs: 1, 3, 5, 7, 9, 11, 13, 15, 17, 34, 42, 44, 45, 46, 50 and 54; and an antisense strand selected from any one of SEQ ID NOs: 58-72.

In one embodiment, a double stranded nucleic acid is provided comprising a sense strand selected from SEQ ID NOs: 1, 3, 5, 7, 9, 11, 13, 15, 17, 34, 42, 44, 45, 46, 50 and 54; and an antisense strand selected from any one of SEQ ID NOs: 58-72.

In one embodiment, a double-stranded nucleic acid is provided consisting of SEQ ID NO:34 and SEQ ID NO:58; SEQ ID NO:34 and SEQ ID NO:59; SEQ ID NO:34 and SEQ ID NO:60; SEQ ID NO:17 and SEQ ID NO:61; SEQ ID NO:34 and SEQ ID NO:62; SEQ ID NO:42 and SEQ ID NO:63; SEQ ID NO:44 and SEQ ID NO:63; SEQ ID NO:45 and SEQ ID NO:63; SEQ ID NO:46 and SEQ ID NO:64; SEQ ID NO:46 and SEQ ID NO:65; SEQ ID NO:46 and SEQ ID NO:66; SEQ ID NO:50 and SEQ ID NO:67; SEQ ID NO:46 and SEQ ID NO:69; SEQ ID NO:54 and SEQ ID NO:70; SEQ ID NO:17 and SEQ ID NO:72, or SEQ ID NO:56 and SEQ ID NO:71.

In one embodiment, a double stranded nucleic acid is provided comprising at least one nucleic acid molecule selected from among SEQ ID NOs: 58-72.

In one embodiment, a double stranded nucleic acid is provided comprising two nucleic acid molecules selected from among SEQ ID NOs: 1-18 or 34-72. In one embodiment, the double stranded nucleic acid comprises a sense strand and an antisense strand.

In one embodiment, each strand of the double stranded nucleic acid has no more than 52 nucleotides.

In one embodiment, a double stranded nucleic acid is provided comprising a sense strand comprising a nucleic acid molecule selected from SEQ ID NOs: 1, 17, 34, 42, 44, 45, 46, 50, 54 and 56; and an antisense strand comprising a nucleic acid molecule selected from any one of SEQ ID NOs: 58-72.

In one embodiment, a double stranded nucleic acid is provided comprising a sense strand comprising a nucleic acid molecule selected from SEQ ID NOs: 1, 3, 5, 7 and 9; and an antisense strand comprising a nucleic acid molecule selected from any one of SEQ ID NOs:58-72.

In one embodiment, the double-stranded nucleic acid comprises nucleic acid molecules comprising SEQ ID NO:34 and SEQ ID NO:58; SEQ ID NO:34 and SEQ ID NO:59; SEQ ID NO:34 and SEQ ID NO:60; SEQ ID NO:17 and SEQ ID NO:61; SEQ ID NO:34 and SEQ ID NO:62; SEQ ID NO:42 and SEQ ID NO:63; SEQ ID NO:44 and SEQ ID NO:63; SEQ ID NO:45 and SEQ ID NO:63; SEQ ID NO:46 and SEQ ID NO:64; SEQ ID NO:46 and SEQ ID NO:65; SEQ ID NO:46 and SEQ ID NO:66; SEQ ID NO:50 and SEQ ID NO:67; SEQ ID NO:46 and SEQ ID NO:69; SEQ ID NO:54 and SEQ ID NO:70; SEQ ID NO:17 and SEQ ID NO:72, or SEQ ID NO:56 and SEQ ID NO:71.

Any of the compositions and uses of siRNA directed to FL2 as described elsewhere herein may utilize any of the foregoing single stranded nucleic acid sequences SEQ ID NOs:58-72, or a double stranded nucleic acids comprising or consisting of any of SEQ ID NOs:58-72.

Any of the nucleic acid sequences described herein may be prepared by any method known in the art, and purified by HPLC or any other method to provide inhibitors suitable for use for the in vitro, ex vivo or in vivo purposes described herein. In some embodiments, the purity of the inhibitor is equal to or greater than 85%. In some embodiment the purity is equal to or greater than 90%. In some embodiments the purity is equal to or greater than 95%. In some embodiment the purity is equal to or greater than 98%. In some embodiments the purity is equal to or greater than 99%. In some embodiments wherein the inhibitor is or comprises a duplex, the purity of the duplex is equal to or greater than 85%. In some embodiments wherein the inhibitor is or comprises a duplex, the purity of the duplex is equal to or greater than 90%. In some embodiments wherein the inhibitor is or comprises a duplex, the purity of the duplex is equal to or greater than 95%. In some embodiments wherein the inhibitor is or comprises a duplex, the purity of the duplex is equal to or greater than 98%. In some embodiments wherein the inhibitor is or comprises a duplex, the purity of the duplex is equal to or greater than 99%. In some embodiments the inhibitor is prepared under current Good Manufacturing Practices. In some embodiments the inhibitor is prepared for human use. In some embodiments the inhibitor is prepare for in vitro or ex vivo use for subsequent administration to humans. In some embodiments the inhibitor is prepared for human administration.

In one aspect, a composition is provided comprising any of the foregoing nucleic acid molecules or double-stranded nucleic acids, and a pharmaceutically acceptable carrier, vehicle, excipient or diluent.

In one embodiment, each strand of the double stranded nucleic acid has no more than 52 nucleotides.

In one embodiment, any one of the foregoing nucleic acids has at least one nucleotide is modified or further modified. In one embodiment, the modified nucleotide is selected from 2′-O-methyl-adenosine, 2′-O-methyl-uridine, 2′-O-methyl-cytosine, 2′-O-methyl-guanosine, 2′-O-methyl-thymidine, 2′-fluoro-adenosine, 2′-fluoro-cytidine, 2′-fluoro-guanosine, 2′-fluoro-uracil, 2′-fluoro-thymidine, deoxycytosine, deoxyguanosine, deoxyadenosine, deoxythymidine, deoxyuridine, a locked adenosine, a locked uridine, a locked guanosine, a locked cytidine, a phosphorothioate, and a phosphodiester cap. In one embodiment, at least one additional nucleotide or modified nucleotide is added to an end of the nucleic acid.

As noted above, locked nucleotides in one embodiment comprise a ribose with a 2′-O, 4′-C methylene bridge, for example, 2′-O, 4′-C methylene adenosine (lA); 2′-O, 4′-C methylene guanosine (lG); 2′-O, 4′-C methylene cytidine (lC); 2′-O, 4′-C methylene uridine (lU); and 2′-O, 4′-C methylene thymine (lT) ribonucleosides. In other embodiments, the locked nucleic acid comprises a methyl group attached to the methylene group. Other types of locked nucleic acids are embraced herein.

In another embodiment, an siRNA of the disclosure comprises at least one 2′-sugar modification. In another embodiment, an siRNA of the disclosure comprises at least one nucleic acid base modification. In another embodiment, an siRNA of the disclosure comprises at least one phosphate backbone modification. As used herein, “at least one” means one or more.

A composition provided in such a kit may be provided in a form suitable for reconstitution prior to use (such as a lyophilized injectable composition) or in a form which is suitable for immediate application to a skin graft or skin grafting site, including to the graft margin, such as a lotion or ointment.

In an embodiment of the disclosure the inhibitor of fidgetin-like 2 is provided by a subcutaneous implant or depot medicament system for the pulsatile delivery of the inhibitor to a skin grafting site to promote skin graft healing.

A medicament in accordance with this aspect of the disclosure may be formulated m any appropriate carrier. Suitable carriers are pharmaceutically acceptable carriers, preferably those consistent with administration topically or administration by injection.

In a non-limiting embodiment, the inhibitor of fidgetin-like 2 is provided in a bulk-eroding system such as polylactic acid and glycolic acid (PLGA) copolymer-based microspheres or microcapsules systems containing the inhibitor of fidgetin-like 2. In an embodiment, blends of PLGA:ethylcellulose systems may be used as an appropriate carrier. A further medicament in accordance with this aspect of the disclosure may be formulated in a surface-eroding system wherein the inhibitor of fidgetin-like 2 is embedded in an erodible matrix such as the poly(ortho) ester and polyanhydride matrices wherein the hydrolysis of the polymer is rapid. A medicament in accordance with this aspect of the disclosure may also be formulated by combining a pulsatile delivery system as described above and an immediate release system such as a lyophilized injectable composition described above.

The inhibitor may be used in a composition with additives. Examples of suitable additives are sodium alginate, as a gelatinizing agent for preparing a suitable base, or cellulose derivatives, such as guar or xanthan gum, inorganic gelatinizing agents, such as aluminum hydroxide or bentonites (termed thixotropic gel-formers), polyacrylic acid derivatives, such as Carbopol®, polyvinylpyrrolidone, microcrystalline cellulose and carboxymethylcellulose. Amphiphilic low molecular weight and higher molecular weight compounds, and also phospholipids, are also suitable. The gels can be present either as water-based hydrogels or as hydrophobic organogels, for example based on mixtures of low and high molecular weight paraffin hydrocarbons and vaseline. The hydrophilic organogels can be prepared, for example, on the basis of high molecular weight polyethylene glycols. These gelatinous forms are washable. Hydrophobic organogels are also suitable. Hydrophobic additives, such as petroleum jelly, wax, oleyl alcohol, propylene glycol monostearate and/or propylene glycol monopalmitostearate, in particular isopropyl myristate can be included. In an embodiment the inhibitor is in a composition comprising one or more dyes, for example yellow and/or red iron oxide and/or titanium dioxide for the purpose of matching as regards color. Compositions may be in any suitable form including gels, lotions, balms, pastes, sprays, powders, bandages, wound dressing, emulsions, creams and ointments of the mixed-phase or amphiphilic emulsion systems (oil/water-water/oil mixed phase), liposomes and transfersomes or plasters/band aid-type coverings. Emulsifiers which can be employed in compositions comprising the inhibitor of fidgetin-like 2 include anionic, cationic or neutral surfactants, for example alkali metal soaps, metal soaps, amine soaps, sulphurated and sulphonated compounds, invert soaps, higher fatty alcohols, partial fatty acid esters of sorbitan and polyoxyethylene sorbitan, e.g. lanette types, wool wax, lanolin or other synthetic products for preparing the oil/water and/or water/oil emulsions.

Compositions comprising the inhibitor of fidgetin-like 2 can also comprise vaseline, natural or synthetic waxes, fatty acids, fatty alcohols, fatty acid esters, for example as monoglycerides, diglycerides or triglycerides, paraffin oil or vegetable oils, hydrogenated castor oil or coconut oil, hog fat, synthetic fats (for example based on caprylic acid, capric acid, lauric acid or stearic acid, such as Softisan®), or triglyceride mixtures, such as Miglyol®, can be used as lipids, in the form of fatty and/or oleaginous and/or waxy components for preparing the ointments, creams or emulsions of the compositions comprising the inhibitor of fidgetin-like 2 used in the methods described herein.

Osmotically active acids and alkaline solutions, for example hydrochloric acid, citric acid, sodium hydroxide solution, potassium hydroxide solution, sodium hydrogen carbonate, may also be ingredients of the compositions and, in addition, buffer systems, such as citrate, phosphate, tris buffer or triethanolamine, for adjusting the pH. It is possible to add preservatives as well, such as methyl benzoate or propyl benzoate (parabens) or sorbic acid, for increasing the stability.

Pastes, powders and solutions are additional forms of compositions comprising the inhibitor of fidgetin-like 2 which can be applied topically. As consistency-imparting bases, the pastes frequently contain hydrophobic and hydrophilic auxiliary substances, preferably, however, hydrophobic auxiliary substances containing a very high proportion of solids. In order to increase dispersity, and also flowability and slipperiness, and also to prevent agglomerates, the powders or topically applicable powders can, for example, contain starch species, such as wheat or rice starch, flame-dispersed silicon dioxide or siliceous earth, which also serve as diluent.

In an embodiment, the compositions comprise further active ingredients suitable for protecting or aiding in healing of the skin graft, for example one or more antibiotics, antiseptics, vitamins, anesthetics, antihistamines, anti-inflammatory agents, moisturizers, penetration-enhancing agents and/or anti-irritants.

In an embodiment of the methods and compositions described herein the subject is a mammal. In an embodiment the subject is human.

Preferably the inhibitor is biomembrane-permeable or is conjugated or otherwise attached to a moiety which renders the inhibitor biomembrane-permeable.

All combinations of the various elements described herein are within the scope of the disclosure unless otherwise indicated herein or otherwise clearly contradicted by context.

The siRNA sequences SEQ ID NO:11 and SEQ ID NO:12 herein are directed to the murine (mouse) orthologue of FL2. The siRNA sequences SEQ ID NO:13 and SEQ ID NO:14 are directed to the rat orthologue of FL2. The siRNA sequences SEQ ID NO:15 and SEQ ID NO:16 are directed to the porcine (pig) orthologue of FL2. Studies in non-human models such as the study described in the example herein are typically conducted with the siRNA that is directed to and inhibits the species-specific FL2 nucleic acid, wherein the siRNA is pharmacologically active. Such siRNAs to other species' FL2 orthologues may be used to treat skin graft healing in such other species (e.g., non-human primates, non-human mammals). In some embodiments, siRNA directed against FL2 of one species can be used in another species, e.g., is cross-reactive with another species, for the uses disclosed herein.

The following siRNA molecules useful for the purposes disclosed herein were prepared.

| RNA sequences | Sequence ID |
|---|---|
| UUACACAGUAUUAAAGCGAUU (sense) | SEQ ID NO: 1 |
| UCGCUUUAAUACUGUGUAAUU (antisense) | SEQ ID NO: 2 |
| CAUCUGAAACCUAGGGUCUUU (sense) | SEQ ID NO: 3 |
| AGACCCUAGGUUUCAGAUGUU (antisense) | SEQ ID NO: 4 |
| GUGACUUAUGCUAGGAGGAUU (sense) | SEQ ID NO: 5 |
| UCCUCCUAGCAUAAGUCACUU (antisense) | SEQ ID NO: 6 |
| GGUCAGAAGCAGAAUGUAUUU (sense) | SEQ ID NO: 7 |
| AUACAUUCUGCUUCUGACCUU (antisense) | SEQ ID NO: 8 |
| CGCCGGCCCACAAGUUGGAdTdT (sense) | SEQ ID NO: 9 |
| UCCAACUUGUGGGCCGGCGdTdT (antisense) | SEQ ID NO: 10 |
| CAGCUCGAGCCCUUUGACAdTdT (sense) | SEQ ID NO: 11 |
| UGUCAAAGGGCUCGAGCUGdTdT (antisense) | SEQ ID NO: 12 |
| CCUCCAACCUCCUCAAGAGdTdT (sense) | SEQ ID NO: 13 |
| CUCUUGAGGAGGUUGGAGGdTdT (antisense) | SEQ ID NO: 14 |
| CGUUGCUGCUCAUCAGCGAdTdT (sense) | SEQ ID NO: 15 |
| UCGCUGAUGAGCAGCAACGdTdT (antisense) | SEQ ID NO: 16 |
| fUfUmA fmAfC AGU AUU AAA GCG ATT (sense) | SEQ ID NO: 17 |
| (Phos) U CGC UUU AAU ACU G UG UAA TT (antisense) | SEQ ID NO: 18 |
| 5′-UUACACAGUAUUAAAGCGATT-3′ (sense) | SEQ ID NO: 34 |
| (Phos) 5′-mUmCGCUUUAAUACUGUGUAATT-3′ (antisense) | SEQ ID NO: 35 |
| (Phos) 5′-mU(s)mC(s)GCUUUAAUACUGUGUAATT-3′ (antisense) | SEQ ID NO: 36 |
| (Phos) 5′-fUfCGCUUUAAUACUGUGUAATT-3′ (antisense) | SEQ ID NO: 37 |
| (Phos) 5′-fU(s)fC(s)GCUUUAAUACUGUGUAATT-3′ (antisense) | SEQ ID NO: 38 |
| (Phos) 5′-mU(s)mC(s)GCUUUAAUAmCfUmGfUmGfUmAmATT-3′ | SEQ ID NO: 39 |
| (antisense) | |
| (Phos) 5′-U(s)CGCUUUAAUACUGUGUAATT-3′ (antisense) | SEQ ID NO: 40 |
| (Phos) 5′-mUfCmGfCmUfUmUAAfUmAfCmUGmUniGfUniAniATT | SEQ ID NO: 41 |
| (antisense) | |
| 5′ mU mU mAmC mAmCmAmGmUmAmUmUmAmAmAmGmCmGmAmUmU3′- | SEQ ID NO: 42 |
| (antisense) | |
| (Phos) 5′-mU mC mGmCmU mU mU mAmAmU mAmCmU mGmU mGmU | SEQ ID NO: 43 |
| mAmAmU mU-3′ (antisense) | |
| 5′ mUmUmAmCmAmCmAmGmUmAmUmUmAmAmAmGdCdGdATT-3′ | SEQ ID NO: 44 |
| (sense) | |
| 5′ mUmUmAmCmAmCmAmGmUmAmUmUmAmAmAmGdCmGmATT-3′ | SEQ ID NO: 45 |
| (sense) | |
| 5′ UUACACAGUAUUAAAGCGA-3′ (sense) | SEQ ID NO: 46 |
| (Phos) 5′-U(s)CGCUUUAAUACUGUGUAATT-3′ (antisense) | SEQ ID NO: 47 |
| (Phos) 5′-UCGCUUUAAUACUGUGUAATT-3′ (antisense) | SEQ ID NO: 48 |
| (Phos) 5′-U(s)C(s)GCUUUAAUACUGUGUAATT-3′ (antisense) | SEQ ID NO: 49 |
| 5′-mUmUAC ACAGUAUUAAAGCGA-3′ (sense) | SEQ ID NO: 50 |
| (Phos) 5′-U(s)CGCUUUAAUACUGUGUmAmATT-3′ (antisense) | SEQ ID NO: 51 |
| (Phos) 5′-UCGCUUUAAUACUGUGUAATT-3′ (antisense) | SEQ ID NO: 52 |
| (Phos) 5′-U(s)C(s)GCUUUAAUACUGUGUAA T(s)T-3′ (antisense) | SEQ ID NO: 53 |
| 5′ lUlUlAlClACAGUAUUAAAGCGATT-3 (sense) | SEQ ID NO: 54 |
| (Phos) 5′-UCGCUUUAAUACUGlUlGlUlAlA TT-3′ (antisense) | SEQ ID NO: 55 |
| 5′ fUfUlAfClACAGUAUUAAAGCGA-3′ (sense) | SEQ ID NO: 56 |
| (Phos) 5′-mU(s)mCmGCUUUAAUACUGUGUAATT-3′ (antisense) | SEQ ID NO: 57 |

wherein d(nucleotide)=deoxy-(nucleotide), m(nucleotide)=2′-O-methyl nucleotide, T=thymidine, f(nucleotide)=2′-fluorodeoxy nucleotide, (Phos)=phosphodiester cap; capital letter nucleotide=RNA nucleotide, l(nucleotide)=a locked nucleotide, and (s)=phosphorothioate. Thus, for example dT represents deoxythymidine, dC represents deoxycytidine, fC represents 2′-fluorodeoxy cytidine ribonucleic acid, fU represents 2′-fluorodeoxy uracil ribonucleic acid, mA represents 2′-O-methyl adenosine ribonucleic acid, mU represents 2′-O-methyl uracil ribonucleic acid, mC represents 2′-O-methyl cytosine ribonucleic acid, and mG represents 2′-O-methyl guanosine ribonucleic acid.

The following siRNA molecules useful for the purposed disclosed herein may be prepared.

| | |
|---|---|
| 5′-fUfCGCUUUAAUACUGUGUAATT-3′ (antisense) | SEQ ID NO: 58 |
| 5′-fU(s)fC(s)GCUUUAAUACUGUGUAATT-3′ (antisense) | SEQ ID NO: 59 |
| 5′-mU(s)mC(s)GCUUUAAUAmCfUmGfUmGfUmAmATT-3′ | SEQ ID NO: 60 |
| (antisense) | |
| 5′-U(s)CGCUUUAAUACUGUGUAATT-3′ (antisense) | SEQ ID NO: 61 |
| 5′-mUfCmGfCmUfUmUAAfUmAfCmUGmUmGfUmAmATT (antisense) | SEQ ID NO: 62 |
| 5′-mU mCmGmCmUmUmUmAmAmUmAmCmUmGmUmGmUmAmAmUmU-3′ | SEQ ID NO: 63 |
| (antisense) | |
| 5′-U(s)CGCUUUAAUACUGUGUAATT-3′ (antisense) | SEQ ID NO: 64 |
| 5′-UCGCUUUAAUACUGUGUAATT-3′ (antisense) | SEQ ID NO: 65 |
| 5′-U(s)C(s)GCUUUAAUACUGUGUAATT-3′ (antisense) | SEQ ID NO: 66 |
| 5′-U(s)CGCUUUAAUACUGUGUmAmATT-3′ (antisense) | SEQ ID NO: 67 |
| 5′-UCGCUUUAAUACUGUGUAATT-3′ (antisense) | SEQ ID NO: 68 |
| 5′-U(s)C(s)GCUUUAAUACUGUGUAA T(s)T-3′ (antisense) | SEQ ID NO: 69 |
| 5′-UCGCUUUAAUACUGlUlGlUlAlA TT-3′ (antisense) | SEQ ID NO: 70 |
| 5′-mU(s)mCmGCUUUAAUACUGUGUAATT-3′ (antisense) | SEQ ID NO: 71 |
| 5′-U CGC UUU AAU ACU G UG UAA TT (antisense) | SEQ ID NO: 72 |

The following shRNA molecule useful for the purposes herein may be prepared:

(SEQ ID NO: 23)
CACCGCTGGAGCCCTTTGACAAGTTCTCGAGAACTTGTCAAAGGGCTCCA
GCTTTT.

The following numbered embodiments, while non-limiting, are exemplary of certain aspects of the disclosure:

1. A method of accelerating or improving the healing of a skin graft or skin grafting site in a subject comprising administering to the subject an amount of an inhibitor of fidgetin-like 2 effective to accelerate healing of the skin graft or skin grafting site.
2. The method of embodiment 1 wherein the remodeling of the skin graft is improved.
3. The method of embodiment 1 wherein the cosmetic appearance of the skin graft is improved.
4. The method of embodiment 1 wherein the skin graft or skin grafting site exhibits a reduced scar formation.
5. The method of embodiment 1 wherein the skin grafting is provided to treat a burn.
6. The method of embodiment 5 wherein the burn is a partial-thickness burn.
7. The method of embodiment 5 wherein the burn is a full-thickness burn.
8. The method of embodiment 5 wherein the skin graft covering a burn comprises a larger total body area burn.
9. The method of embodiment 1 wherein the skin grafting is provided to treat an injury.
10. The method of embodiment 9 wherein the injury is a large open wound.
11. The method of embodiment 9 wherein the injury is an ulcer.
12. The method of embodiment 11 wherein the ulcer is a bedsore.
13. The method of embodiment 9 wherein the injury is a skin infection.
14. The method of embodiment 9 wherein the injury is a result from skin cancer surgery.
15. The method of embodiment 1 wherein the skin grafting is provided to cover a larger surface area than available from donor skin.
16. The method of embodiment 1 wherein the skin graft has a large meshing ratio.
17. The method of embodiment 16 wherein the meshing ratio is greater than 1.5 to 1.
18. The method of embodiment 16 wherein the meshing ratio is 3:1.
19. The method of embodiment 5 wherein the meshing ratio is 9:1.
20. The method of embodiment 1 wherein the skin graft is a split-thickness skin graft.
21. The method of embodiment 1 wherein the skin graft is a full-thickness skin graft.
22. The method of embodiment 1 wherein the inhibitor of fidgetin-like 2 is an siRNA or a shRNA directed to fidgetin-like 2.
23. The method of embodiment 22, wherein the siRNA is administered.
24. The method of embodiment 22 wherein the shRNA is administered.
25. The method of embodiment 23 wherein the siRNA directed against a DNA or RNA encoding human fidgetin-like 2 has at least one 2′ sugar modification.
26. The method of embodiment 24, wherein the shRNA directed against a DNA or RNA encoding human fidgetin-like 2 has at least one 2′ sugar modification.
27. The method of embodiment 1, wherein the siRNA or shRNA is directed against an mRNA encoding the human fidgetin-like 2.
28. The method of embodiment 27, wherein the siRNA comprises a sequence set forth in SEQ

ID NOs:1-18 or 34-72.

29. The method of embodiment 28 wherein the siRNA consists of a double-stranded nucleic acid selected from among SEQ ID NO:1 and SEQ ID NO: 2; SEQ ID NO:3 and SEQ ID NO: 4; SEQ ID NO:5 and SEQ ID NO: 6; SEQ ID NO:7 and SEQ ID NO: 8; SEQ ID NO:9 and SEQ ID NO: 10; SEQ ID NO:11 and SEQ ID NO: 12; SEQ ID NO:13 and SEQ ID NO: 14; SEQ ID NO:15 and SEQ ID NO: 16; SEQ ID NO:17 and SEQ ID NO:18; SEQ ID NO:34 and SEQ ID NO:35; SEQ ID NO:34 and SEQ ID NO:36; SEQ ID NO:34 and SEQ ID NO:37; SEQ ID NO:34 and SEQ ID NO:38; SEQ ID NO:34 and SEQ ID NO:39; SEQ ID NO:17 and SEQ ID NO:40; SEQ ID NO:34 and SEQ ID NO:41; SEQ ID NO:42 and SEQ ID NO:43; SEQ ID NO:44 and SEQ ID NO:43; SEQ ID NO:45 and SEQ ID NO:43; SEQ ID NO:46 and SEQ ID NO:47; SEQ ID NO:46 and SEQ ID NO:48; SEQ ID NO:46 and SEQ ID NO:49; SEQ ID NO:50 and SEQ ID NO:51; SEQ ID NO:46 and SEQ ID NO:53; SEQ ID NO:54 and SEQ ID NO:55; SEQ ID NO:56 and SEQ ID NO:57; SEQ ID NO:34 and SEQ ID NO:58; SEQ ID NO:34 and SEQ ID NO:59; SEQ ID NO:34 and SEQ ID NO:60; SEQ ID NO:17 and SEQ ID NO:61; SEQ ID NO:34 and SEQ ID NO:62; SEQ ID NO:42 and SEQ ID NO:63; SEQ ID NO:44 and SEQ ID NO:63; SEQ ID NO:45 and SEQ ID NO:63; SEQ ID NO:46 and SEQ ID NO:64; SEQ ID NO:46 and SEQ ID NO:65; SEQ ID NO:46 and SEQ ID NO:66; SEQ ID NO:50 and SEQ ID NO:67; SEQ ID NO:46 and SEQ ID NO:69; SEQ ID NO:54 and SEQ ID NO:70; SEQ ID NO:17 and SEQ ID NO:72, or SEQ ID NO:56 and SEQ ID NO:71.
30. The method of embodiment 28 wherein the siRNA comprises a double-stranded nucleic acid selected from among SEQ ID NO:1 and SEQ ID NO: 2; SEQ ID NO:3 and SEQ ID NO: 4; SEQ ID NO:5 and SEQ ID NO: 6; SEQ ID NO:7 and SEQ ID NO: 8; SEQ ID NO:9 and SEQ ID NO: 10; SEQ ID NO:11 and SEQ ID NO: 12; SEQ ID NO:13 and SEQ ID NO: 14; SEQ ID NO:15 and SEQ ID NO: 16; SEQ ID NO:17 and SEQ ID NO: 18; SEQ ID NO:17 and SEQ ID NO:18; SEQ ID NO:34 and SEQ ID NO:35; SEQ ID NO:34 and SEQ ID NO:36; SEQ ID NO:34 and SEQ ID NO:37; SEQ ID NO:34 and SEQ ID NO:38; SEQ ID NO:34 and SEQ ID NO:39; SEQ ID NO:17 and SEQ ID NO:40; SEQ ID NO:34 and SEQ ID NO:41; SEQ ID NO:42 and SEQ ID NO:43; SEQ ID NO:44 and SEQ ID NO:43; SEQ ID NO:45 and SEQ ID NO:43; SEQ ID NO:46 and SEQ ID NO:47; SEQ ID NO:46 and SEQ ID NO:48; SEQ ID NO:46 and SEQ ID NO:49; SEQ ID NO:50 and SEQ ID NO:51; SEQ ID NO:46 and SEQ ID NO:53; SEQ ID NO:54 and SEQ ID NO:55; SEQ ID NO:56 and SEQ ID NO:57; SEQ ID NO:34 and SEQ ID NO:58; SEQ ID NO:34 and SEQ ID NO:59; SEQ ID NO:34 and SEQ ID NO:60; SEQ ID NO:17 and SEQ ID NO:61; SEQ ID NO:34 and SEQ ID NO:62; SEQ ID NO:42 and SEQ ID NO:63; SEQ ID NO:44 and SEQ ID NO:63; SEQ ID NO:45 and SEQ ID NO:63; SEQ ID NO:46 and SEQ ID NO:64; SEQ ID NO:46 and SEQ ID NO:65; SEQ ID NO:46 and SEQ ID NO:66; SEQ ID NO:50 and SEQ ID NO:67; SEQ ID NO:46 and SEQ ID NO:69; SEQ ID NO:54 and SEQ ID NO:70; SEQ ID NO:17 and SEQ ID NO:72, or SEQ ID NO:56 and SEQ ID NO:71.
31. The method of embodiment 1 wherein the inhibitor is topically applied to the skin grafting site.
32. A method of accelerating or improving the healing of a skin graft or skin grafting site in a subject comprising directly administering to the skin graft or skin grafting site an amount of a siRNA or shRNA directed against a DNA or RNA encoding a human Fidgetin like-2 comprising the amino acid set forth in SEQ ID NO:20 effective to inhibit scarring.
33. The method of embodiment 24 wherein the shRNA consists of SEQ ID NO:23.
34. The method of embodiment 24 wherein the shRNA comprises SEQ ID NO:23.

This disclosure will be better understood from the Experimental Details, which follow.

However, one skilled in the art will readily appreciate that the specific methods and results discussed are merely illustrative of the disclosure as described more fully in the claims that follow thereafter.

### EXPERIMENTAL DETAILS

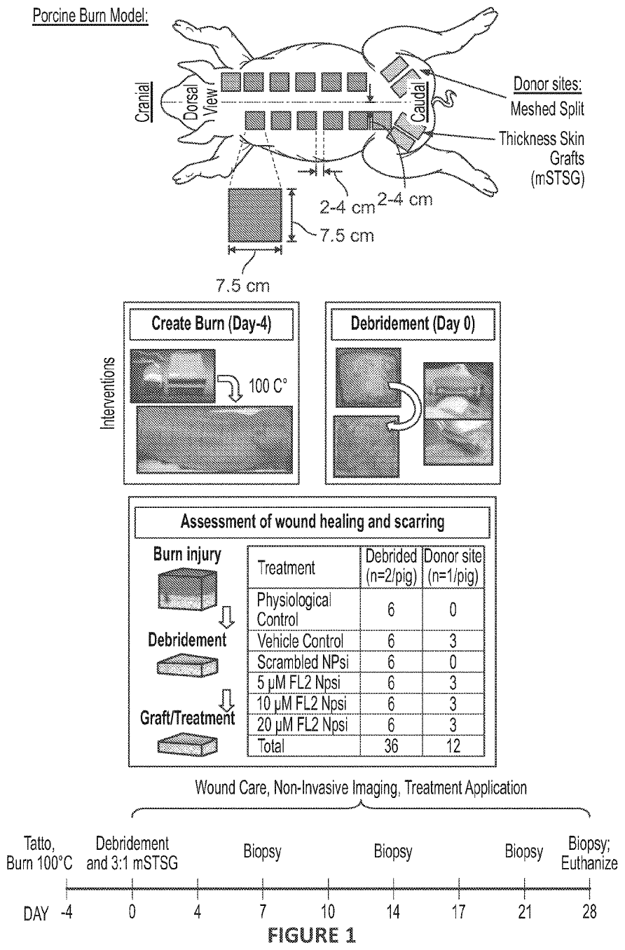

A porcine burn model was used to study the healing of skin grafts. Ten 5×5 cm burns were created on the dorsum of anesthetized Yorkshire pig. The schematic in FIG. 1 indicates the timeline and methods utilized throughout the study. Non-invasive measurements to include digital, Silhouette, and laser speckle imaging.

Meshed split thickness skin grafts (mSTSG) were used.

The nanoparticle siRNA (NPsi) are manufactured using a water-in-oil 118 emulsion whereby the aqueous FL2-targeting or scramble (control) siRNA solution is encapsulated by Zonyl® FSO 119 and polyethylene oxide-polypropylene oxide polymer. The siRNA tested had the sequence sense strand: CGUUGCUGCUCAUCAGCGA[dT][dT] (SEQ ID NO:15) and antisense strand: UCGCUGAUGAGCAGCAACG[dT][dT] (SEQ ID NO:16). A scrambled siRNA sequence served as the control. Doses tested: 5, 10, 20 μM FL2 NPsi or 20 μM scrambled NPsi (n=2).

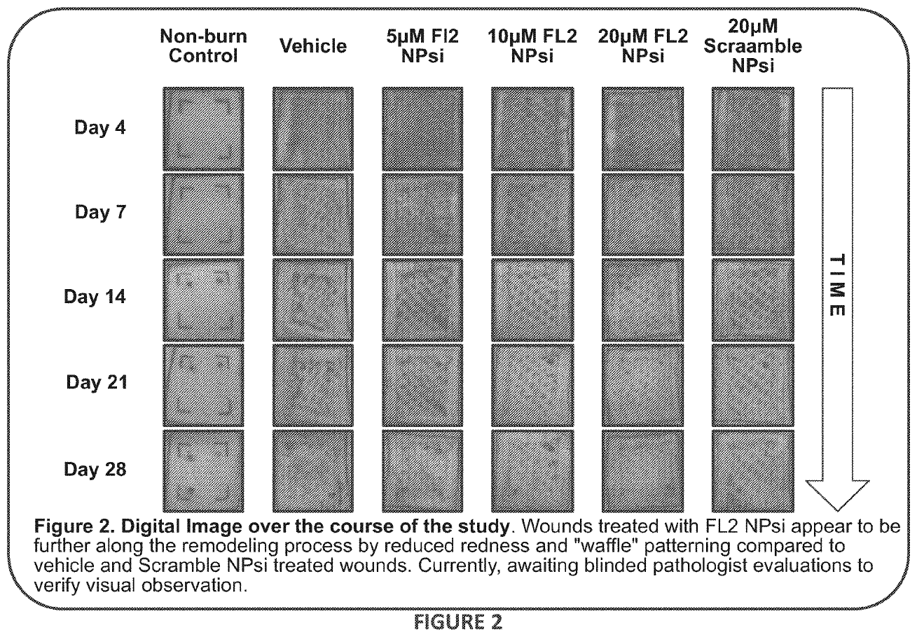

FIG. 2 depicts digital images of the healing sites. The study showed that topical application of NP FL2 siRNA increased the rate of healing of large ratio mSTSG skin grafts.

1. A method of accelerating or improving the healing of a skin graft or skin grafting site in a subject comprising administering to the subject an amount of an inhibitor of fidgetin-like 2 effective to accelerate healing of the skin graft or skin grafting site.

2. The method of claim 1 wherein the remodeling of the skin graft is improved.

3. The method of claim 1 wherein the cosmetic appearance of the skin graft is improved.

4.-14. (canceled)

15. The method of claim 1 wherein the skin grafting is provided to cover a larger surface area than available from donor skin.

16. The method of claim 1 wherein the skin graft has a large meshing ratio.

17. The method of claim 16 wherein the meshing ratio is greater than 1.5 to 1.

18. The method of claim 16 wherein the meshing ratio is 3:1.

19. The method of claim 5 wherein the meshing ratio is 9:1.

20. The method of claim 1 wherein the skin graft is a split-thickness skin graft.

21. The method of claim 1 wherein the skin graft is a full-thickness skin graft.

22. The method of claim 1 wherein the inhibitor of fidgetin-like 2 is an siRNA or a shRNA directed against fidgetin-like 2.

23.-24. (canceled)

25. The method of claim 22 wherein the siRNA or shRNA directed against a DNA or RNA encoding human fidgetin-like 2 has at least one 2′ sugar modification.

26. (canceled)

27. The method of claim 1, wherein the siRNA or shRNA is directed against an mRNA encoding the human fidgetin-like 2.

28. The method of claim 27, wherein the siRNA comprises a sequence set forth in SEQ ID NOs:1-18 or 34-72.

29. The method of claim 28 wherein the siRNA consists of a double-stranded nucleic acid selected from among SEQ ID NO:1 and SEQ ID NO: 2; SEQ ID NO:3 and SEQ ID NO: 4; SEQ ID NO:5 and SEQ ID NO: 6; SEQ ID NO:7 and SEQ ID NO: 8; SEQ ID NO:9 and SEQ ID NO: 10; SEQ ID NO:11 and SEQ ID NO: 12; SEQ ID NO:13 and SEQ ID NO: 14; SEQ ID NO:15 and SEQ ID NO: 16; SEQ ID NO:17 and SEQ ID NO: 18; SEQ ID NO:17 and SEQ ID NO:18; SEQ ID NO:34 and SEQ ID NO:35; SEQ ID NO:34 and SEQ ID NO:36; SEQ ID NO:34 and SEQ ID NO:37; SEQ ID NO:34 and SEQ ID NO:38; SEQ ID NO:34 and SEQ ID NO:39; SEQ ID NO:17 and SEQ ID NO:40; SEQ ID NO:34 and SEQ ID NO:41; SEQ ID NO:42 and SEQ ID NO:43; SEQ ID NO:44 and SEQ ID NO:43; SEQ ID NO:45 and SEQ ID NO:43; SEQ ID NO:46 and SEQ ID NO:47; SEQ ID NO:46 and SEQ ID NO:48; SEQ ID NO:46 and SEQ ID NO:49; SEQ ID NO:50 and SEQ ID NO:51; SEQ ID NO:46 and SEQ ID NO:53; SEQ ID NO:54 and SEQ ID NO:55; SEQ ID NO:56 and SEQ ID NO:57; SEQ ID NO:34 and SEQ ID NO:58; SEQ ID NO:34 and SEQ ID NO:59; SEQ ID NO:34 and SEQ ID NO:60; SEQ ID NO:17 and SEQ ID NO:61; SEQ ID NO:34 and SEQ ID NO:62; SEQ ID NO:42 and SEQ ID NO:63; SEQ ID NO:44 and SEQ ID NO:63; SEQ ID NO:45 and SEQ ID NO:63; SEQ ID NO:46 and SEQ ID NO:64; SEQ ID NO:46 and SEQ ID NO:65; SEQ ID NO:46 and SEQ ID NO:66; SEQ ID NO:50 and SEQ ID NO:67; SEQ ID NO:46 and SEQ ID NO:69; SEQ ID NO:54 and SEQ ID NO:70; SEQ ID NO:17 and SEQ ID NO:72, or SEQ ID NO:56 and SEQ ID NO:71.

30. The method of claim 28 wherein the siRNA comprises a double-stranded nucleic acid selected from among SEQ ID NO:1 and SEQ ID NO: 2; SEQ ID NO:3 and SEQ ID NO: 4; SEQ ID NO:5 and SEQ ID NO: 6; SEQ ID NO:7 and SEQ ID NO: 8; SEQ ID NO:9 and SEQ ID NO: 10; SEQ ID NO:11 and SEQ ID NO: 12; SEQ ID NO:13 and SEQ ID NO: 14; SEQ ID NO:15 and SEQ ID NO: 16; SEQ ID NO:17 and SEQ ID NO: 18; SEQ ID NO:17 and SEQ ID NO:18; SEQ ID NO:34 and SEQ ID NO:35; SEQ ID NO:34 and SEQ ID NO:36; SEQ ID NO:34 and SEQ ID NO:37; SEQ ID NO:34 and SEQ ID NO:38; SEQ ID NO:34 and SEQ ID NO:39; SEQ ID NO:17 and SEQ ID NO:40; SEQ ID NO:34 and SEQ ID NO:41; SEQ ID NO:42 and SEQ ID NO:43; SEQ ID NO:44 and SEQ ID NO:43; SEQ ID NO:45 and SEQ ID NO:43; SEQ ID NO:46 and SEQ ID NO:47; SEQ ID NO:46 and SEQ ID NO:48; SEQ ID NO:46 and SEQ ID NO:49; SEQ ID NO:50 and SEQ ID NO:51; SEQ ID NO:46 and SEQ ID NO:53; SEQ ID NO:54 and SEQ ID NO:55; SEQ ID NO:56 and SEQ ID NO:57; SEQ ID NO:34 and SEQ ID NO:58; SEQ ID NO:34 and SEQ ID NO:59; SEQ ID NO:34 and SEQ ID NO:60; SEQ ID NO:17 and SEQ ID NO:61; SEQ ID NO:34 and SEQ ID NO:62; SEQ ID NO:42 and SEQ ID NO:63; SEQ ID NO:44 and SEQ ID NO:63; SEQ ID NO:45 and SEQ ID NO:63; SEQ ID NO:46 and SEQ ID NO:64; SEQ ID NO:46 and SEQ ID NO:65; SEQ ID NO:46 and SEQ ID NO:66; SEQ ID NO:50 and SEQ ID NO:67; SEQ ID NO:46 and SEQ ID NO:69; SEQ ID NO:54 and SEQ ID NO:70; SEQ ID NO:17 and SEQ ID NO:72, or SEQ ID NO:56 and SEQ ID NO:71.

31. The method of claim 1 wherein the inhibitor is topically applied to the skin grafting site.

32. A method of accelerating or improving the healing of a skin graft or skin grafting site in a subject comprising directly administering to the skin graft or skin grafting site an amount of a siRNA or shRNA directed against a DNA or RNA encoding a human Fidgetin like-2 comprising the amino acid set forth in SEQ ID NO:20 effective to inhibit scarring.

33. The method of claim 22 wherein the shRNA consists of SEQ ID NO:23.

34. The method of claim 22 wherein the shRNA comprises SEQ ID NO:23.


SEQUENCE LISTING (72 sequences)

SEQ ID NO: 1 - DNA, 21 bp, Artificial Sequence
  siRNA to human fidgetin-like 2, sense strand
uuacacaguauuaaagcgauu

SEQ ID NO: 2 - DNA, 21 bp, Artificial Sequence
  siRNA to human fidgetin-like 2, antisense strand
ucgcuuuaauacuguguaauu

SEQ ID NO: 3 - DNA, 21 bp, Artificial Sequence
  siRNA to human fidgetin-like 2, sense strand
caucugaaaccuagggucuuu

SEQ ID NO: 4 - DNA, 21 bp, Artificial Sequence
  siRNA to human fidgetin-like 2, antisense strand
agacccuagguuucagauguu

SEQ ID NO: 5 - DNA, 21 bp, Artificial Sequence
  siRNA to human fidgetin-like 2, sense strand
gugacuuaugcuaggaggauu

SEQ ID NO: 6 - DNA, 21 bp, Artificial Sequence
  siRNA to human fidgetin-like 2, antisense strand
uccuccuagcauaagucacuu

SEQ ID NO: 7 - DNA, 21 bp, Artificial Sequence
  siRNA to human fidgetin-like 2, sense strand
ggucagaagcagaauguauuu

SEQ ID NO: 8 - DNA, 21 bp, Artificial Sequence
  siRNA to human fidgetin-like 2, antisense strand
auacauucugcuucugaccuu

SEQ ID NO: 9 - DNA, 21 bp, Artificial Sequence
  siRNA to human fidgetin-like 2, sense strand
  misc_feature (20)..(20): n is deoxythymidine
  misc_feature (21)..(21): n is deoxythymidine
cgccggcccacaaguuggann

SEQ ID NO: 10 - DNA, 21 bp, Artificial Sequence
  siRNA to human fidgetin-like 2, antisense strand
  misc_feature (20)..(20): n is deoxythymidine
  misc_feature (21)..(21): n is deoxythymidine
uccaacuugugggccggcgnn

SEQ ID NO: 11 - DNA, 21 bp, Artificial Sequence
  siRNA to mouse fidgetin-like 2, sense strand
  misc_feature (20)..(20): n is deoxythymidine
  misc_feature (21)..(21): n is deoxythymidine
cagcucgagcccuuugacann

SEQ ID NO: 12 - DNA, 21 bp, Artificial Sequence
  siRNA to mouse fidgetin-like 2, antisense strand
  misc_feature (20)..(20): n is deoxythymidine
  misc_feature (21)..(21): n is deoxythymidine
ugucaaagggcucgagcugnn

SEQ ID NO: 13 - DNA, 21 bp, Artificial Sequence
  siRNA to rat fidgetin-like 2, sense strand
  misc_feature (20)..(20): n is deoxythymidine
  misc_feature (21)..(21): n is deoxythymidine
ccuccaaccuccucaagagnn

SEQ ID NO: 14 - DNA, 21 bp, Artificial Sequence
  siRNA to rat fidgetin-like 2, antisense strand
  misc_feature (20)..(20): n is deoxythymidine
  misc_feature (21)..(21): n is deoxythymidine
cucuugaggagguuggaggnn

SEQ ID NO: 15 - DNA, 21 bp, Artificial Sequence
  siRNA to pig fidgetin-like 2, sense strand
  misc_feature (20)..(20): n is deoxythymidine
  misc_feature (21)..(21): n is deoxythymidine
cguugcugcucaucagcgann

SEQ ID NO: 16 - DNA, 21 bp, Artificial Sequence
  siRNA to pig fidgetin-like 2, sense antisense strand
  misc_feature (20)..(20): n is deoxythymidine
  misc_feature (21)..(21): n is deoxythymidine
ucgcugaugagcagcaacgnn

SEQ ID NO: 17 - DNA, 21 bp, Artificial Sequence
  siRNA to human fidgetin-like 2, sense strand
  misc_feature (1)..(1): n is 2′-fluoruridine
  misc_feature (2)..(2): n is 2′-fluorouridine
  misc_feature (3)..(3): n is 2′-O-methyladenosine
  misc_feature (4)..(4): n is 2′fluorocytidine
  misc_feature (5)..(5): n is 2′O-methyladenosine
  misc_feature (6)..(6): n is 2′fluorocytidine
  misc_feature (6)..(6): n is 2′fluorodeoxycytidine
nnnnnnaguauuaaagcgatt

SEQ ID NO: 18 - DNA, 21 bp, Artificial Sequence
  siRNA to human fidgetin-like 2, antisense strand 3′ end has a phosphodiester cap
ttaaugugucauaauuucgcu

SEQ ID NO: 19 - DNA, 4711 bp, Human
agtgagctatggggacactactgcactgtagcctgggcaacagagcaagaccttgtctcaaaaatgtatatatattttgggctttttttcctaaaacgggaactacaacagcatatttgcgagctgatgagagtgacccagcagagagggaaatggatcagctctgttgaagatgcactggacaccagaacacgcccagcccctcaaccagtggccagagcagcacctggacgtctcctccaccaccccgtcgccggcccacaagttggagttgccccctgggggtcgccaacgctgccactacgcttgggcacacgacgacatctcagccctcactgcctccaacctcctaaagcgctatgcagagaagtactctggggtcttggattctccctacgagcgtccggccctgggcgggtacagcgacgcctccttcctcaacggcgccaaaggggatcccgagccctggccagggccggagccaccctaccccttggcctcactccacgaaggcctcccaggaaccaaatcgggcggtggcggcggttccggggccctggggggctccccagttttagccgggaacctccctgaacccctctacgccggcaatgcgtgcgggggcccatcggcggcgcccgagtacgcggccggctacggcggggggtacctggcgccgggttactgcgcgcagacgggcgccgcgctgcccccgccgcccccggccgcgctcctgcagcccccaccgcctccggggtacgggccctcagcgccgctgtacaactatcccgcagggggctacgcagcgcagcccggctatggcgcgctcccgccgcccccaggcccacccccggccccctacctgaccccgggcctgcccgcgcccacgcccctgcccgcgccggcaccgcccaccgcctatggcttccccacggccgcgccgggtgccgaatccgggctgtcgctgaagcgcaaggccgccgacgaggggcccgagggccgctaccgcaagtacgcgtacgagcccgccaaggcccccgtggctgacggagcctcctaccccgccgcggacaacggcgaatgtcggggcaacgggttccgggccaagccgccaggagccgcggaggaggcgtcgggcaagtacggtggcggcgtccccctcaaggtcctgggctcccccgtctacggcccgcaactggagccctttgaaaagttcccggagcgggccccggctcctcgtggggggttcgccgtgccgtcgggggagactcccaaaggcgtggaccctggggccctggagctggtgacgagcaagatggtggactgcgggcccccggtgcagtgggcggatgtggcgggccagggcgcgctcaaggcggcgctggaggaggagctggtgtggcccctgctcaggccgcccgcctacccgggcagcctgcgcccgccgcggaccgtcctgctctttgggccgcggggcgcgggcaaagcgctgctgggccgctgcctcgccacgcagctgggcgccacgctgttgcgcctgcgcggcgcgaccctggctgcgcccggcgccgccgagggcgcgcgcctcctccaggccgccttcgcggccgcgcgctgccgcccaccctccgtactcctcatcagcgagctagaggcgctgctccccgcccgggacgacggcgcggcggcagggggcgcgctgcaggtgccgctcctggcctgcctggacgggggctgcggcgcgggggctgacggcgtgctggttgtgggcaccacctcgcggcccgcggctctggacgaggcgacccgccggcgcttctctctccgcttctacgtggcgctgcccgacagcccggcccgcgggcagatcctgcagcgggcgctggcccagcagggctgcgcgctcagtgagcgggaactggcggcgctggtgcagggcacgcagggcttctctgggggcgagctggggcagctgtgccagcaggcggcggccggggcgggcctcccggggctgcagcgccccctctcctacaaggacctggaggcggcgctggccaaggtgggccctagggcctctgccaaggaactggactcgttcgtggagtgggacaaaatgtacggctccggacactgacggcgcgcgggggaggccgcgggagccgcagtccctccgtccccgccgcctccgcgtgggagggatgtcactgactaaacccggctggcaggggctggagtggtgaatgtgggatcggggacaggaggggtctgccggtggatattttttttttcgtgggaaggaaaatgcttctgccaggcagatgccatatgcgccgtgtactcaggtttttcctatttattgtggactggaagctcgccatctccgcccggcagaccgggcagatccggcatgggctggcacccggggccttaagaactcctgctctcttgccacaacgcttttgtctcctcgctatctgaatggcaccctccttctccctcactctctccatcccattctctgcattctcttggttttctctcccttttgctttgtcgctgacacccctgcccaccccatgctggccctgtttctctcctgcccctccctccccagctctccatccctcaccctctgtgcttctgtctccatccctggctctccagcgtccctggccttttggtccctgagctttaatgcctttccctgccttctgttcttatttggactgcagtggccctttgcaggagctctggaggcccaggggctgaggaggagggttacccctctacccatctgaaacctagggtctagggggatcaaggaaaaaaagtccccaaagaaggggaattttttgtttgtttttgaggggagatcccagaaatgtagcttgtttcatattttagtcttcttatttttgtaaaatgtgtagaatttgctgtttttctttttcttttgacaactcaggaagaaactgacctcagaaagaatgttagactttggctgctctcctgtgtgcccctcacacctgccccctcccccccactccatccaggggaccaaattctcccagacactcaaaaaatgagacttacggggaaggggagaggaagacccagaggcctcagtgaaaccccagctattcctggtcagaagcagaatgtattcctaagggcttcctccccagggccgaggcctaggcatgaatgtggggagtgggctgtggggtttgagagaagggaggccttattcctctcctgctgctccccaccccctgccccacccaacccctccgctgagtgttttctgtgaagggctatccagagttaggatgcccttgcccaattccttcctgagacccagaaggtagggtgggagggcccaaatgggaaggtgacctaagcagaaagtctccagaaaggtcatgtcccctggccctgccttggcagaggtccccagtgacttatgctaggaggattccatctgggtagacagtctggccacaaaatcagctactggacctcagccatctctgctggaggctctgaggaggagtgagcatccctcacttgtgggggctctgtgaggaaatgtgccttccccattcccccggagtcctaggtctggagctccagggctgggagagggtgagggagatgggcaggggtgttttctctgaccttgggggcttagtctcagtcctgcctgaactttccactaggcttggaacccttccaagaaccatatttctctccttcccaccaattttcccttgatgaggctttagcagtttgctcccaccacccccagcccatttcacaactctgatcttagtccaaagcaggggacacgcccccccaccaccactttttctctctcccatctcagcctcctgtgcagttccttgcctgcccgtgcatttcctagagtctactgcctcccccctggctgggagggtgtctgggggggatctttcaggggccctggcacccagggcctgtgctggcctaggagtgctgaccagaaggctgctctgttcccccccacccccgttgctttctggccccctctttggagccagccacccacagggctttggtgcctcagaagcagtgggctgccgggtcacagccgcaggctgcaaaagaccctcggagggagcatggagtgaggggttctctctcaggtgtgtatgtattggggggtgggggtgggtggagggtgtcagggaagttggggtgggatcccagccttcccttcaagaggcagggagctctgggaggtggagtccccaccgctttctctactaggctcctcctgttccccaggcttggggagctttgcacaaggagactgcccccagcctagtggcacctacctcatgggctctggggcaggtaggggaagggccagtccagctctggtaatgctggggggaggcataccaaagaatccaggggcagggagtggggagggtgacttccgagctggcctctccccttcctctacccagactggggctgggatcctctcctcccgctgtaaccatttctacctcattttgctgcgtgttgtacatggacgtatttatctcctgtctgacgatgctctgcagttgtggtctgtctacctcagaagagactgtattttaaaagaaagtattacacagtattaaagcgatgacatgtggtttgcaaaaaaaaaaaaaaaaaa

SEQ ID NO: 20 - protein, 653 aa, Human
MHWTPEHAQPLNQWPEQHLDVSSTTPSPAHKLELPPGGRQRCHYAWAHDDISALTASNLLKRYAEKYSGVLDSPYERPALGGYSDASFLNGAKGDPEPWPGPEPPYPLASLHEGLPGTKSGGGGGSGALGGSPVLAGNLPEPLYAGNACGGPSAAPEYAAGYGGGYLAPGYCAQTGAALPPPPPAALLQPPPPPGYGPSAPLYNYPAGGYAAQPGYGALPPPPGPPPAPYLTPGLPAPTPLPAPAPPTAYGFPTAAPGAESGLSLKRKAADEGPEGRYRKYAYEPAKAPVADGASYPAADNGECRGNGFRAKPPGAAEEASGKYGGGVPLKVLGSPVYGPQLEPFEKFPERAPAPRGGFAVPSGETPKGVDPGALELVTSKMVDCGPPVQWADVAGQGALKAALEEELVWPLLRPPAYPGSLRPPRTVLLFGPRGAGKALLGRCLATQLGATLLRLRGATLAAPGAAEGARLLQAAFAAARCRPPSVLLISELEALLPARDDGAAAGGALQVPLLACLDGGCGAGADGVLVVGTTSRPAALDEATRRRFSLRFYVALPDSPARGQILQRALAQQGCALSERELAALVQGTQGFSGGELGQLCQQAAAGAGLPGLQRPLSYKDLEAALAKVGPRASAKELDSFVEWDKMYGSGH

SEQ ID NO: 21-22 - skipped (2 entries)

SEQ ID NO: 23 - DNA, 56 bp, Artificial Sequence
  shRNA to human fidgetin-like 2
caccgctggagccctttgacaagttctcgagaacttgtcaaagggctccagctttt

SEQ ID NO: 24-33 - skipped (10 entries)

SEQ ID NO: 34 - DNA, 21 bp, Artificial Sequence
  siRNA to human fidgetin-like 2, sense strand
uuacacaguauuaaagcgatt

SEQ ID NO: 35 - DNA, 21 bp, Artificial Sequence
  siRNA to human fidgetin-like 2, antisense strand 5′ phosphodiester cap
  misc_feature (1)..(1): n is 2′-O-methyluridine
  misc_feature (2)..(2): n is 2′O′methylcytidine
nngcuuuaauacuguguaatt

SEQ ID NO: 36 - DNA, 21 bp, Artificial Sequence
  siRNA to human fidgetin-like 2, sense strand 5′ phosphodiester cap Phosphorothioate bond between nucleotides 1 and 2 and between nucleotides 2 and 3
  misc_feature (1)..(1): n is 2′-O′methyuridine
  misc_feature (1)..(2): phosphorotioate linkage
  misc_feature (1)..(1): 5 phosphodiester cap
  misc_feature (2)..(3): phosphorotioate linkage
  misc_feature (2)..(2): n is 2′-O-methylcytidine
nngcuuuaauacuguguaatt

SEQ ID NO: 37 - DNA, 21 bp, Artificial Sequence
  siRNA to human fidgetin-like 2, antisense strand 5′ phosphodiester cap
  misc_feature (1)..(1): n is 2′-fluorodeoxyuridine
  misc_feature (1)..(1): 5′ phosphodiester cap
  misc_feature (2)..(2): n is 2′-fluorodeoxycytidine
nngcuuuaauacuguguaatt

SEQ ID NO: 38 - DNA, 21 bp, Artificial Sequence
  siRNA to human fidgetin-like 2, sense strand 5′ phosphodiester cap Phosphorothioate bond between nucleotides 1 and 2 and between nucleotides 2 and 3
  misc_feature (1)..(1): 2′-fluorodeoxyuridine
  misc_feature (1)..(1): 5′ phosphodiester cap
  misc_feature (1)..(2): phosphorothioate bond
  misc_feature (2)..(3): phosphorothioate bond
  misc_feature (2)..(2): 2′-fluorodeoxycytidine
nngcuuuaauacuguguaatt

SEQ ID NO: 39 - DNA, 21 bp, Artificial Sequence
  siRNA to human fidgetin-like 2, antisense strand 5′ phosphodiester cap Phosphorothioate bond between nucleotides 1 and 2 and between nucleotides 2 and 3
  misc_feature (1)..(1): 1 n is 2′-O-methyluridine
  misc_feature (1)..(1): 5′ phosphodiester cap
  misc_feature (1)..(2): phosphorothioate bond
  misc_feature (2)..(3): phosphorothioate bond
  misc_feature (2)..(2): n is 2′-O-methylcytidine
  misc_feature (12)..(12): n is 2′-O-methylcytidine
  misc_feature (13)..(13): n is 2′-fluorodeoxyuridine
  misc_feature (14)..(14): n is 2′-O-methylguanosine
  misc_feature (15)..(15): n is 2′-fluorodeoxyuridine
  misc_feature (16)..(16): n is 2′-O-methylguanosine
  misc_feature (17)..(17): n is 2′-fluorodeoxyuridine
  misc_feature (18)..(18): n is 2′-O′-methyladenosine
  misc_feature (19)..(19): n is 2′-O′-methyladenosine
nngcuuuaauannnnnnnntt

SEQ ID NO: 40 - DNA, 21 bp, Artificial Sequence
  siRNA to human fidgetin-like 2, sense strand 5′ phosphodiester cap Phosphorothioate bond between nucleotides 1 and 2
  misc_feature (1)..(1): 5′ phosphodiester cap
  misc_feature (1)..(2): phosphorotioate bond
ucgcuuuaauacuguguaatt

SEQ ID NO: 41 - DNA, 21 bp, Artificial Sequence
  siRNA to human fidgetin-like 2, antisense strand 5′ phosphodiester cap
  misc_feature (1)..(1): n is 2′-O-methyluridine
  misc_feature (1)..(1): 5′ phosphodiester cap
  misc_feature (2)..(2): n is 2′-fluorodeoxycytidine
  misc_feature (3)..(3): n is 2′-O-methylguanosine
  misc_feature (4)..(4): n is 2′-fluorodeoxycytidine
  misc_feature (5)..(5): n is 2′-O-methyluridine
  misc_feature (6)..(6): n is 2′-fluorodeoxyuridine
  misc_feature (7)..(7): n is 2′-O-methyluridine
  misc_feature (10)..(10): n is 2′-fluorodeoxyuridine
  misc_feature (11)..(11): n is 2′-O′-methyladenosine
  misc_feature (12)..(12): n is 2′-fluorodeoxycytidine
  misc_feature (13)..(13): n is 2′-O-methyluridine
  misc_feature (15)..(15): n is 2′-O-methyluridine
  misc_feature (16)..(16): n is 2′-O-methylguanosine
  misc_feature (17)..(17): n is 2′-fluorodeoxyuridine
  misc_feature (18)..(18): n is 2′-O′-methyladenosine
  misc_feature (19)..(19): n is 2′-O′-methyladenosine
nnnnnnnaannnngnnnnntt

SEQ ID NO: 42 - DNA, 21 bp, Artificial Sequence
  siRNA to human fidgetin-like 2, sense strand
  misc_feature (1)..(1): n is 2′-O-methyluridine
  misc_feature (2)..(2): n is 2′-O-methyluridine
  misc_feature (3)..(3): n is 2′-O′-methyladenosine
  misc_feature (4)..(4): n is 2′-O-methylcytidine
  misc_feature (5)..(5): n is 2′-O′-methyladenosine
  misc_feature (6)..(6): n is 2′-O-methylcytidine
  misc_feature (7)..(7): n is 2′-O′-methyladenosine
  misc_feature (8)..(8): n is 2′-O-methylguanosine
  misc_feature (9)..(9): n is 2′-O-methyluridine
  misc_feature (10)..(10): n is 2′-O′-methyladenosine
  misc_feature (11)..(11): n is 2′-O-methyluridine
  misc_feature (12)..(12): n is 2′-O-methyluridine
  misc_feature (13)..(13): n is 2′-O′-methyladenosine
  misc_feature (14)..(14): n is 2′-O′-methyladenosine
  misc_feature (15)..(15): n is 2′-O′-methyladenosine
  misc_feature (16)..(16): n is 2′-O-methylguanosine
  misc_feature (17)..(17): n is 2′-O-methylcytidine
  misc_feature (18)..(18): n is 2′-O-methylguanosine
  misc_feature (19)..(19): n is 2′-O′-methyladenosine
  misc_feature (20)..(20): n is 2′-O-methyluridine
  misc_feature (21)..(21): n is 2′-O-methyluridine
nnnnnnnnnnnnnnnnnnnnn

SEQ ID NO: 43 - DNA, 20 bp, Artificial Sequence
  siRNA to human fidgetin-like 2, antisense strand 5′ phosphodiester cap
  misc_feature (1)..(1): n is 2′-O-methyluridine
  misc_feature (1)..(1): 5′ phosphodiester cap
  misc_feature (2)..(2): n is 2′-O-methylcytidine
  misc_feature (3)..(3): n is 2′-O-methylguanosine
  misc_feature (4)..(4): n is 2′-O-methylcytidine
  misc_feature (5)..(5): n is 2′-O-methyluridine
  misc_feature (6)..(6): n is 2′-O-methyluridine
  misc_feature (7)..(7): n is 2′-O-methyluridine
  misc_feature (8)..(8): n is 2′-O′-methyladenosine
  misc_feature (9)..(9): n is 2′-O′-methyladenosine
  misc_feature (10)..(10): n is 2′-O-methyluridine
  misc_feature (11)..(11): n is 2′-O′-methyladenosine
  misc_feature (12)..(12): n is 2′-O-methylcytidine
  misc_feature (13)..(13): n is 2′-O-methyluridine
  misc_feature (14)..(14): n is 2′-O-methylguanosine
  misc_feature (15)..(15): n is 2′-O-methyluridine
  misc_feature (16)..(16): n is 2′-O-methylguanosine
  misc_feature (17)..(17): n is 2′-O-methyluridine
  misc_feature (18)..(18): n is 2′-O′-methyladenosine
  misc_feature (19)..(19): n is 2′-O′-methyladenosine
  misc_feature (20)..(20): n is 2′-O-methyluridine
nnnnnnnnnnnnnnnnnnnn

SEQ ID NO: 44 - DNA, 21 bp, Artificial Sequence
  siRNA to human fidgetin-like 2, sense strand
  misc_feature (1)..(1): n is 2′-O-methyluridine
  misc_feature (2)..(2): n is 2′-O-methyluridine
  misc_feature (3)..(3): n is 2′-O′-methyladenosine
  misc_feature (4)..(4): n is 2′-O-methylcytidine
  misc_feature (5)..(5): n is 2′-O′-methyladenosine
  misc_feature (6)..(6): n is 2′-O-methylcytidine
  misc_feature (7)..(7): n is 2′-O′-methyladenosine
  misc_feature (8)..(8): n is 2′-O-methylguanosine
  misc_feature (9)..(9): n is 2′-O-methyluridine
  misc_feature (10)..(10): n is 2′-O′-methyladenosine
  misc_feature (11)..(11): n is 2′-O-methyluridine
  misc_feature (12)..(12): n is 2′-O-methyluridine
  misc_feature (13)..(13): n is 2′-O′-methyladenosine
  misc_feature (14)..(14): n is 2′-O′-methyladenosine
  misc_feature (15)..(15): n is 2′-O′-methyladenosine
  misc_feature (16)..(16): n is 2′-O-methylguanosine
  misc_feature (17)..(17): n is deoxycytidine
  misc_feature (18)..(18): n is deoxyguanosine
  misc_feature (19)..(19): n is deoxyadenosine
nnnnnnnnnnnnnnnnnnntt

SEQ ID NO: 45 - DNA, 21 bp, Artificial Sequence
  siRNA to human fidgetin-like 2, antisense strand
  misc_feature (1)..(1): n is 2′-O-methyluridine
  misc_feature (2)..(2): n is 2′-O-methyluridine
  misc_feature (3)..(3): n is 2′-O′-methyladenosine
  misc_feature (4)..(4): n is 2′-O-methylcytidine
  misc_feature (5)..(5): n is 2′-O′-methyladenosine
  misc_feature (6)..(6): n is 2′-O-methylcytidine
  misc_feature (7)..(7): n is 2′-O′-methyladenosine
  misc_feature (8)..(8): n is 2′-O-methylguanosine
  misc_feature (9)..(9): n is 2′-O-methyluridine
  misc_feature (10)..(10): n is 2′-O′-methyladenosine
  misc_feature (11)..(11): n is 2′-O-methyluridine
  misc_feature (12)..(12): n is 2′-O-methyluridine
  misc_feature (13)..(13): n is 2′-O′-methyladenosine
  misc_feature (14)..(14): n is 2′-O′-methyladenosine
  misc_feature (15)..(15): n is 2′-O′-methyladenosine
  misc_feature (16)..(16): n is 2′-O-methylguanosine
  misc_feature (17)..(17): n is deoxycytidine
  misc_feature (18)..(18): n is 2′-O-methylguanosine
  misc_feature (19)..(19): n is 2′-O′-methyladenosine
nnnnnnnnnnnnnnnnnnntt

SEQ ID NO: 46 - DNA, 19 bp, Artificial Sequence
  siRNA to human fidgetin-like 2, sense strand
uuacacaguauuaaagcga

SEQ ID NO: 47 - DNA, 21 bp, Artificial Sequence
  siRNA to human fidgetin-like 2, antisense strand 5′ phosphodiester cap Phosphorothioate bond between nucleotides 1 and 2
  misc_feature (1)..(1): 5′ phosphodiester cap
  misc_feature (1)..(2): phosphorothioate bond
ucgcuuuaauacuguguaatt

SEQ ID NO: 48 - DNA, 21 bp, Artificial Sequence
  siRNA to human fidgetin-like 2, sense strand 5′ phosphodiester cap
  misc_feature (1)..(1): 5′ phosphodiester cap
ucgcuuuaauacuguguaatt

SEQ ID NO: 49 - DNA, 21 bp, Artificial Sequence
  siRNA to human fidgetin-like 2, antisense strand 5′ phosphodiester cap Phosphorothioate bond between nucleotides 1 and 2 and between nucleotides 2 and 3
  misc_feature (1)..(1): 5′ phosphodiester cap
  misc_feature (1)..(2): phosphorothioate bond
  misc_feature (2)..(3): phosphorothioate bond
ucgcuuuaauacuguguaatt

SEQ ID NO: 50 - DNA, 19 bp, Artificial Sequence
  siRNA to human fidgetin-like 2, sense strand
  misc_feature (1)..(1): n is 2′-O-methyluridine
  misc_feature (2)..(2): n is 2′-O-methyluridine
nnacacaguauuaaagcga

SEQ ID NO: 51 - DNA, 21 bp, Artificial Sequence
  siRNA to human fidgetin-like 2, antisense strand 5′ phosphodiester cap Phosphorothioate bond between nucleotides 1 and 2
  misc_feature (1)..(1): 5′ phosphodiester cap
  misc_feature (1)..(2): phosphorothioate bond
  misc_feature (18)..(18): n is 2′-O′-methyladenosine
  misc_feature (19)..(19): n is 2′-O′-methyladenosine
ucgcuuuaauacugugunntt

SEQ ID NO: 52 - DNA, 21 bp, Artificial Sequence
  siRNA to human fidgetin-like 2, sense strand 5′ phosphodiester cap
  misc_feature (1)..(1): 5′ phosphodiester cap
ucgcuuuaauacuguguaatt

SEQ ID NO: 53 - DNA, 21 bp, Artificial Sequence
  siRNA to human fidgetin-like 2, antisense strand 5′ phosphodiester cap Phosphorothioate bond between nucleotides 1 and 2 and between nucleotides 2 and 3 and between nucleotides 19 and 20
  misc_feature (1)..(1): 5′ phosphodiester cap
  misc_feature (1)..(2): phosphorothioate bond
  misc_feature (2)..(3): phosphorothioate bond
  misc_feature (19)..(20): phosphorothioate bond
ucgcuuuaauacuguguaatt

SEQ ID NO: 54 - DNA, 21 bp, Artificial Sequence
  siRNA to human fidgetin-like 2, sense strand
  misc_feature (1)..(1): n is locked uridine
  misc_feature (2)..(2): n is locked uridine
  misc_feature (3)..(3): n is locked adenosine
  misc_feature (4)..(4): n is locked cytidine
  misc_feature (5)..(5): n is locked adenosine
nnnnncaguauuaaagcgatt

SEQ ID NO: 55 - DNA, 21 bp, Artificial Sequence
  siRNA to human fidgetin-like 2, antisense strand 5′ phosphodiester cap
  misc_feature (1)..(1): 5′ phosphodiester cap
  misc_feature (15)..(15): n is locked uridine
  misc_feature (16)..(16): n is locked guanosine
  misc_feature (17)..(17): n is locked uridine
  misc_feature (18)..(18): n is locked adenosine
  misc_feature (19)..(19): n is locked adenosine
ucgcuuuaauacugnnnnntt

SEQ ID NO: 56 - DNA, 19 bp, Artificial Sequence
  siRNA to human fidgetin-like 2, sense strand
  misc_feature (1)..(1): n is 2′-fluorodeoxyuridine
  misc_feature (2)..(2): n is 2′-fluorodeoxyuridine
  misc_feature (3)..(3): n is locked adenosine
  misc_feature (4)..(4): n is 2′-fluorodeoxycytidine
  misc_feature (5)..(5): n is locked adenosine
nnnnncaguauuaaagcga

SEQ ID NO: 57 - DNA, 21 bp, Artificial Sequence
  siRNA to human fidgetin-like 2, antisense strand 5′ phosphodiester cap Phosphorothioate bond between nucleotides 1 and 2
  misc_feature (1)..(1): n is 2′-O-methyluridine
  misc_feature (1)..(1): 5′ phosphodiester cap
  misc_feature (1)..(2): phosphorothioate bond
  misc_feature (2)..(2): n is 2′-O-methylcytidine
  misc_feature (3)..(3): n is 2′-O-methylguanosine
nnncuuuaauacuguguaatt

SEQ ID NO: 58 - DNA, 21 bp, Artificial Sequence
  siRNA to human fidgetin-like 2, antisense strand
  misc_feature (1)..(1): n is 2′-fluorodeoxydeoxyuridine
  misc_feature (2)..(2): n is 2′-fluorodeoxydeoxycytidine
nngcuuuaauacuguguaatt

SEQ ID NO: 59 - DNA, 21 bp, Artificial Sequence
  iRNA to human fidgetin-like 2, antisense strand Phosphorothioate bond between nucleotides 1 and 2 and between nucleotides 2 and 3
  misc_feature (1)..(1): n is 2′-fluorodeoxydeoxyuridine
  misc_feature (2)..(2): n is 2′-fluorodeoxydeoxycytidine
nngcuuuaauacuguguaatt

SEQ ID NO: 60 - DNA, 21 bp, Artificial Sequence
  siRNA to human fidgetin-like 2, antisense strand Phosphorothioate bond between nucleotides 1 and 2 and between nucleotides 2 and 3
  misc_feature (1)..(1): 1 n is 2′-O-methyluridine
  misc_feature (2)..(2): n is 2′-O-methylcytidine
  misc_feature (12)..(12): n is 2′-O-methylcytidine
  misc_feature (13)..(13): n is 2′-fluorodeoxydeoxyuridine
  misc_feature (14)..(14): n is 2′-O-methylguanosine
  misc_feature (15)..(15): n is 2′-fluorodeoxydeoxyuridine
  misc_feature (16)..(16): n is 2′-O-methylguanosine
  misc_feature (17)..(17): n is 2′-fluorodeoxydeoxyuridine
  misc_feature (18)..(18): n is 2′-O-methyladenosine
  misc_feature (19)..(19): n is 2′-O-methyladenosine
nngcuuuaauannnnnnnntt

SEQ ID NO: 61 - DNA, 21 bp, Artificial Sequence
  siRNA to human fidgetin-like 2, antisense strand Phosphorothioate bond between nucleotides 1 and 2
ucgcuuuaauacuguguaatt

SEQ ID NO: 62 - DNA, 21 bp, Artificial Sequence
  siRNA to human fidgetin-like 2, antisense strand
  misc_feature (1)..(1): n is 2′-O-methyluridine
  misc_feature (2)..(2): n is 2′-fluorodeoxydeoxycytidine
  misc_feature (3)..(3): n is 2′-O-methylguanosine
  misc_feature (4)..(4): n is 2′-fluorodeoxydeoxycytidine
  misc_feature (5)..(5): n is 2′-O-methyluridine
  misc_feature (6)..(6): n is 2′-fluorodeoxydeoxyuridine
  misc_feature (7)..(7): n is 2′-O-methyluridine
  misc_feature (10)..(10): n is 2′-fluorodeoxydeoxyuridine
  misc_feature (11)..(11): n is 2′-O-methyladenosine
  misc_feature (12)..(12): n is 2′-fluorodeoxydeoxycytidine
  misc_feature (13)..(13): n is 2′-O-methyluridine
  misc_feature (15)..(15): n is 2′-O-methyluridine
  misc_feature (16)..(16): n is 2′-O-methylguanosine
  misc_feature (17)..(17): n is 2′-fluorodeoxydeoxyuridine
  misc_feature (18)..(18): n is 2′-O-methyladenosine
  misc_feature (19)..(19): n is 2′-O-methyladenosine
nnnnnnnaannnngnnnnntt

SEQ ID NO: 63 - DNA, 20 bp, Artificial Sequence
  siRNA to human fidgetin-like 2, antisense strand
  misc_feature (1)..(1): n is 2′-O-methyluridine
  misc_feature (2)..(2): n is 2′-O-methylcytidine
  misc_feature (3)..(3): n is 2′-O-methylguanosine
  misc_feature (4)..(4): n is 2′-O-methylcytidine
  misc_feature (5)..(5): n is 2′-O-methyluridine
  misc_feature (6)..(6): n is 2′-O-methyluridine
  misc_feature (7)..(7): n is 2′-O-methyluridine
  misc_feature (8)..(8): n is 2′-O-methyladenosine
  misc_feature (9)..(9): n is 2′-O-methyladenosine
  misc_feature (10)..(10): n is 2′-O-methyluridine
  misc_feature (11)..(11): n is 2′-O-methyladenosine
  misc_feature (12)..(12): n is 2′-O-methylcytidine
  misc_feature (13)..(13): n is 2′-O-methyluridine
  misc_feature (14)..(15): n is a, c, g, or t
  misc_feature (16)..(16): n is 2′-O-methylguanosine
  misc_feature (17)..(17): n is 2′-O-methyluridine
  misc_feature (18)..(18): n is 2′-O-methyladenosine
  misc_feature (19)..(19): n is 2′-O-methyladenosine
  misc_feature (20)..(20): n is 2′-O-methyluridine
nnnnnnnnnnnnnnnnnnnn

SEQ ID NO: 64 - DNA, 21 bp, Artificial Sequence
  siRNA to human fidgetin-like 2, antisense strand Phosphorothioate bond between nucleotides 1 and 2
ucgcuuuaauacuguguaatt

SEQ ID NO: 65 - DNA, 21 bp, Artificial Sequence
  siRNA to human fidgetin-like 2, antisense strand
ucgcuuuaauacuguguaatt

SEQ ID NO: 66 - DNA, 21 bp, Artificial Sequence
  siRNA to human fidgetin-like 2, antisense strand Phosphorothioate bond between nucleotides 1 and 2 and between nucleotides 2 and 3
ucgcuuuaauacuguguaatt

SEQ ID NO: 67 - DNA, 21 bp, Artificial Sequence
  siRNA to human fidgetin-like 2, antisense strand Phosphorothioate bond between nucleotides 1 and 2
  misc_feature (18)..(18): n is 2′-O-methyladenosine
  misc_feature (19)..(19): n is 2′-O-methyladenosine
ucgcuuuaauacugugunntt

SEQ ID NO: 68 - DNA, 21 bp, Artificial Sequence
  siRNA to human fidgetin-like 2, antisense strand
ucgcuuuaauacuguguaatt

SEQ ID NO: 69 - DNA, 21 bp, Artificial Sequence
  siRNA to human fidgetin-like 2, antisense strand Phosphorothioate bond between nucleotides 1 and 2 and between nucleotides 2 and 3 and between nucleotides 19 and 20
ucgcuuuaauacuguguaatt

SEQ ID NO: 70 - DNA, 21 bp, Artificial Sequence
  siRNA to human fidgetin-like 2, antisense strand
  misc_feature (15)..(15): n is locked uridine
  misc_feature (16)..(16): n is locked guanosine
  misc_feature (17)..(17): n is locked uridine
  misc_feature (18)..(18): n is locked adenosine
  misc_feature (19)..(19): n is locked adenosine
ucgcuuuaauacugnnnnntt

SEQ ID NO: 71 - DNA, 21 bp, Artificial Sequence
  siRNA to human fidgetin-like 2, antisense strand Phosphorothioate bond between nucleotides 1 and 2
  misc_feature (1)..(1): n is 2′-O-methyluridine
  misc_feature (2)..(2): n is 2′-O-methylcytidine
  misc_feature (3)..(3): n is 2′-O-methylguanosine
nnncuuuaauacuguguaatt

SEQ ID NO: 72 - DNA, 21 bp, Artificial Sequence
  siRNA to human fidgetin-like 2, antisense strand
ucgcuuuaauacuguguaatt